(19) United States
(12) Patent Application Publication
(10) Pub. No.: US 2009/0275632 A1
(43) Pub. Date: Nov. 5, 2009

(54) Methods of diagnosis and treatment

(75) Inventor: Manel Esteller, Madrid (ES)

Correspondence Address: FOLEY AND LARDNER LLP;SUITE 500, 3000 K STREET NW, WASHINGTON, DC, 20007, US

(73) Assignee: OncoMethylome Sciences SA

(21) Appl. No.: 11/976,914

(22) Filed: Oct. 29, 2007

Related U.S. Application Data
(60) Provisional application No. 60/854,726, filed on Oct. 27, 2006.

(51) Int. Cl. A61K 31/7105; C12Q 1/68; C40B 30/04; C12N 15/00

(52) U.S. Cl. 514/44 A; 435/6; 506/9; 435/320.1; 514/44 R

(57) ABSTRACT

A method of diagnosing a disease in which an miRNA is expressed at a lower level comprises, in a test sample obtained from a subject, determining the methylation status of at least one gene encoding the miRNA in the sample, wherein the presence of methylation is indicative of the presence of the disease. Methylation of the gene causes a down regulation in expression which may also be monitored. Related methods and kits are also described based upon methylation of miRNA encoding genes. The primary miRNA of interest is 124a miRNA.

### CROSS-REFERENCE TO RELATED APPLICATION

This application claims priority to U.S. Provisional Patent Application No. 60/854,726, filed on Oct. 27, 2006. The contents of that application is incorporated herein by reference in its entirety.

### FIELD OF THE INVENTION

The present invention relates to diagnosis and therapy. In particular, the invention relates to cancer diagnosis and treatment. More particularly, the invention relates to micro RNA molecules (miRNA), especially miR-124a, and the role that methylation of genes encoding miRNAs plays in disease.

### BACKGROUND OF THE INVENTION

MicroRNAs (miRNAs) are short, approximately 22 nucleotide, non-coding RNAs that are thought to regulate gene expression by sequence-specific base pairing in the 3′ unstranslated regions of the targets mRNA inducing direct mRNA degradation or translational inhibition (1,2). miRNA expression patterns can be developmentally regulated, tissue-specific or steadily expressed in the whole organism (1,2) and are considered to play important roles in cell proliferation, apoptosis and differentiation (1,2). In disease, recent studies have shown that miRNA expression profiles are distinct between normal tissues and the derived-tumors (3) and between different tumor types (3). Interestingly, downregulation of subsets of miRNAs is a common finding in many of these studies (2,3), suggesting that some of these miRNAs may act as putative tumor suppressor genes.

This last indication has been studied in more detail for particular cases, and for example the down-regulated let-7, miR-15/miR-16 and miR-127 target the oncogenic factors RAS, BCL-2 and BCL-6, respectively (4-6). One explanation is a failure at the post-transcriptional regulation of these microRNAs in cancer cells (7). However, additional mechanisms could also be invoked. In this regard, restoration of microRNA-127 expression in cancer cells by treatment with a DNA demethylating agent in combination with a histone deacetylase inhibitor has been recently reported (6 & 8). However, the gene encoding this micro RNA was shown to be heavily methylated both in normal cells and in cancer cells.

### DESCRIPTION OF THE INVENTION

The present invention is based around the discovery of a direct link between methylation of a gene encoding an miRNA and cancer. In particular, methylation of the genes encoding miR-124a has been shown to be functionally linked to a down-regulation of this miRNA in cancer cells, with a consequential effect on oncogene activity. This link between methylation status of an miRNA encoding gene and cancer is useful in a number of diagnostic and therapeutic applications.

Accordingly, in a first aspect the invention provides a method of diagnosing a disease in which an miRNA expression profile is altered comprising, in a test sample obtained from a subject, determining the methylation status of at least one gene/DNA encoding the miRNA in the sample, wherein the presence of methylation, in particular hypermethylation, is indicative of (a positive diagnosis of) the presence of the disease.

Preferably, the miRNA is expressed at a lower level in the disease condition than in normal subjects. Methylation causes down regulation of miRNA expression from its own promoter and thus can be used as a diagnostic indicator of the disease.

“Diagnosis” is defined herein to include monitoring the state and progression of the disease, checking for recurrence of disease following treatment and monitoring the success of a particular treatment. The tests may also have prognostic value, and this is included within the definition of the term “diagnosis”. The prognostic value of the tests may be used as a marker of potential susceptibility to the disease in question. Thus patients at risk may be identified before the disease has a chance to manifest itself in terms of symptoms identifiable in the patient.

According to a further aspect, the invention provides a method for predicting the likelihood of successful treatment of a disease, in which an miRNA expression profile is altered, with a DNA demethylating agent and/or a DNA methyltransferase inhibitor and/or a HDAC inhibitor comprising determining the methylation status of at least one gene/DNA encoding the miRNA in a sample obtained from a subject, wherein if the gene/DNA is methylated, in particular hypermethylated, the likelihood of successful treatment is higher than if the gene/DNA is unmethylated or methylated at a lower level (i.e. not hypermethylated). Preferably, the miRNA is expressed at a lower level in the disease condition than in normal subjects.

The opposite scenario is also envisaged in the present invention. Thus, in a related aspect, the invention provides a method for predicting the likelihood of resistance to treatment of a disease in which an miRNA expression profile is altered with a DNA demethylating agent and/or a DNA methyltransferase inhibitor and/or a HDAC inhibitor comprising determining the methylation status of at least one gene/DNA encoding the miRNA in a sample obtained from a subject, wherein if the gene/DNA is unmethylated, or methylated at a lower level (i.e. not hypermethylated) the likelihood of resistance to treatment is higher than if the gene/DNA is methylated, in particular hypermethylated. Preferably, the miRNA is expressed at a lower level in the disease condition than in normal subjects.

Since methylation of the 124a miRNA genes manifests itself in down regulation of gene expression, the invention also provides a method of selecting a suitable treatment regimen for a disease in which an miRNA is expressed at a lower level (or in which the expression profile is altered) comprising determining the methylation status of at least one gene encoding the miRNA in a sample obtained from a subject, wherein if the gene is methylated, in particular hypermethylated, a DNA demethylating agent and/or a DNA methyltransferase inhibitor and/or a HDAC inhibitor is selected for treatment.

The opposite scenario is also envisaged in the present invention. Thus, in a related aspect, the detection of an unmethylated, or methylated at a lower level (i.e. not hypermethylated) miRNA gene, preferably one or more 124a miRNA genes, indicates that treatment with a DNA demethylating agent and/or a DNA methyltransferase inhibitor and/or a HDAC inhibitor is contra-indicated. Accordingly, alternative treatments should be explored.

In each of the above methods, confirmation of the effect of the DNA demethylating agent and/or a DNA methyltransferase inhibitor and/or a HDAC inhibitor may be sought by further contacting the sample with the DNA demethylating agent and/or a DNA methyltransferase inhibitor and/or a HDAC inhibitor and determining whether the methylation status of the one or more miRNA genes is altered.

By “likelihood of successful treatment” is meant the probability that treatment of the disease using any one or more of the listed therapeutic agents will be successful.

“Successful treatment” is defined to include complete recovery and also significant regression of the disease, and improved survival rates. Improved alleviation of symptoms may also be considered as “successful treatment” in the present invention.

“Resistance” is defined as a reduced probability that treatment of the disease will be successful using any one of the specified therapeutic agents and/or that higher doses will be required to achieve a therapeutic effect.

By “DNA demethylating agent” is meant any molecule, compound or otherwise which is capable of reducing, removing or inhibiting methylation. In particular, methylation of one or more genes encoding a disease associated miRNA (such as 124a miRNA) is inhibited by the DNA demethylating agent. This increases expression of the miRNA.

By “DNA methyltransferase inhibitor” is meant any molecule, compound or otherwise which is capable of inhibiting DNA methyltransferase activity and thus acting to up-regulate expression of the miRNA which is down-regulated in the relevant disease condition. DNA methyltransferases catalyze transfer of a methyl group to DNA. In particular, DNA methyltransferases are responsible for methylation of DNA at CpG sites, which can cause down regulation of important tumour suppressor genes, such as the genes encoding 124a miRNA. Although not bound by this theory, inhibiting DNA methyltransferase activity is thought to prevent down regulation of important tumour suppressor genes through methylation, thus leading to the effective treatment of diseases such as cancer. Inhibitors of DNA methyltransferase activity may influence expression of the DNA methyltransferases or may inhibit the enzymatic activity of the protein for example.

“HDAC inhibitor” is defined as any molecule, compound or otherwise capable of inhibiting histone deacetylase activity and thus acting to up-regulate expression of the miRNA which is down-regulated in the relevant disease condition. Histone deacetylases are a class of enzymes which are responsible for removal of acetyl groups from lysine amino acids in histones. This removal of an acetyl group means that the positively charged lysine becomes available for interaction with DNA. The interaction of histones with DNA generally down regulates gene expression by blocking access to the DNA of components required for transcription. Many HDAC inhibitors are in clinical trials and are known to be useful in treating cancer. Inhibitors of HDAC activity may influence expression of the HDACs or may inhibit the enzymatic activity of the protein for example.

By “methylation status” is meant the level of methylation of cytosine residues (found in CpG pairs) in the relevant miRNA gene which are relevant to the regulation of gene expression. Thus, the levels of methylation of an miRNA gene are determined by any suitable means in order to reflect whether the gene is likely to be down regulated or not. Generally, an increase in methylation is associated with a corresponding decrease in gene expression.

“Expression levels”, unless otherwise stated, are defined to include levels of mRNA produced by transcription of the appropriate miRNA-encoding gene. Changes in the level of expression may be measured directly or indirectly. Indirect measurement may involve determining expression of genes, at either the mRNA or protein level, whose expression is modified or at least partially determined by the relevant miRNA.

“Hypermethylation” is a well-known term in the art. It is defined as an increase in the level of methylation above normal levels. Thus, it relates to aberrant methylation at specific CpG sites in a gene, often in the promoter region. The terms “methylation” and “hypermethylation” may be utilised interchangeably herein. Normal levels of methylation may be defined by comparison to non-diseased cells for example. Methylation and hypermethylation are generally linked to down regulation of gene expression. In this invention, methylation and in particular hypermethylation of specific miRNA genes, such as the 124a miRNA genes, is indicative of a loss of expression of these important miRNA products and thus provides a reliable indicator of the disease condition.

“Suitable treatment regimen” is defined to include the choice of treatment which is to be made by the individual carrying out the method. The regimen chosen is one deemed suitable on the basis of the methylation status and/or expression levels of the relevant miRNA gene and is selected from a DNA demethylating agent and/or a DNA methyltransferase inhibitor and/or a HDAC inhibitor according to the invention. Of course, as would be readily appreciated by the skilled practitioner, the specific dosage regime may be calculated according to the body surface area of the patient or the volume of body space to be occupied, dependent on the particular route of administration to be used. The amount of the composition actually administered will, however, be determined by a medical practitioner based on the circumstances pertaining to the disorder to be treated, such as the severity of the symptoms, the age, weight and response of the individual.

All methods of the invention may be carried out with respect to any gene encoding an miRNA, and whose expression is altered due to methylation or hypermethylation in a disease condition. Thus, for the first time herein methylation of miRNA encoding genes has been shown to be disease associated. Particularly preferred is the 124a miRNA and the genes encoding this molecule which are shown herein for the first time to be methylated and thus the miRNA is down-regulated in various cancer types.

The terms miR-124a and 124a miRNA are used interchangeably herein. This particular miRNA molecule is encoded by three separate genes located at three different chromosomal locations within the human genome. The approved gene symbol (as per the HUGO gene nomenclature committee, see www.gene.ucl.ac.uk/nomenclature) for each of the three genes is MIRN124A1, MIRN124A2 and MIRN124A3 respectively. The corresponding approved gene names are microRNA 124a-1, microRNA 124a-2 and microRNA 124a-3 respectively. The respective chromosomal locations for these genes is 8p23.1, 8q12.3 and 20q13.33 respectively. Aliases include hsa-mir-124a-1, hsa-mir-124a-2 and hsa-mir-124a-3 respectively. Thus, reference to “one or more genes encoding 124a miRNA” is intended to cover these genes. Of course, as appropriate, the skilled person would appreciate that functionally relevant variants of each of the gene sequences may also be detected according to the methods of the invention. For example, the methylation status of a number of splice variants may be determined according to the methods of the invention. Variant sequences preferably have at least 90%, at least 91%, at least 92%, at least 93%, at least 94%, at least 95%, at least 96%, at least 97%, at least 98%, or at least 99% nucleotide sequence identity with the nucleotide sequences in the database entries. Computer programs for determining percentage nucleotide sequence identity are available in the art, including the Basic Local Alignment Search Tool (BLAST) available from the National Center for Biotechnology Information. The miR-124a/124a miRNA molecule has the following nucleotide sequence:

| | |
|---|---|
| ACCGUAAGUGGCGCACGGAAUU. | (SEQ ID NO: 1) |

All of these methods are most preferably in vitro methods carried out on an isolated sample. In one embodiment, the methods may also include the step of obtaining the sample.

In a most preferred embodiment, the subject is a human subject. Generally the subject may be a patient wherein a potential disease, in particular cancer, has been identified and the method may be used to determine if indeed there is a potentially dangerous condition and also to guide treatment depending upon the methylation status of the one or more miRNA encoding gene(s).

The test sample is generally any sample taken preferably from the subject under test in which the methylation status of the at least one miRNA encoding genes is reflective of the disease status. The sample may comprise a tissue sample taken from the subject which is suspected of being a tumor, wherein the disease is preferably cancer. The tissue chosen may be determined by the type of cancer which is suspected or is to be treated. Preferred cancer types according to the methods of the invention are discussed in more detail below and the skilled person would immediately appreciate which type of sample would be appropriate depending upon the cancer concerned (for example suitable colon samples for diagnosing colon cancer).

However, any other suitable test sample in which methylation status of at least one gene encoding an miRNA, most preferably the 124a miRNA gene, can be determined to indicate the presence of disease or the likelihood of successful treatment of the disease with the specified agents or indeed to select a suitable treatment for the disease are included within the scope of the invention. Test samples for diagnostic, prognostic, or personalised medicinal uses may be obtained from surgical samples, such as biopsies or fine needle aspirates, from paraffin embedded tissues or from a body fluid for example. Non-limiting examples of samples which may be used, as appropriate, in the present invention include whole blood, serum, plasma, urine, chyle, stool, ejaculate, sputum, nipple aspirate and saliva.

All of these aspects of the invention rely upon determining the methylation status of at least one gene encoding an miRNA, preferably 124a miRNA.

The methylation status is determined for the promoter region of the gene or genes encoding the miRNA. The methylation status may, however, (also or alternatively) be determined in introns and exons as appropriate. Thus, methylation of the gene, and often the promoter of the gene, encoding the miRNA causes down regulation of expression of the miRNA which is associated with the disease. Preferably, the methylation status of at least one defined CpG island, which may be found (at least partially) in the promoter region, of the gene encoding the diagnostically useful miRNA is determined. CpG islands in an miRNA encoding gene can be readily determined utilising the CpG Island Search Program for example (see http://cpgislands.usc.edu/ and Takai D, Jones P A. The CpG island searcher: a new WWW resource. In Silico Biol. 3, 235-240 (2003)).

Most preferably, the methods of the invention include determining the methylation status of at least one of the genes encoding 124a miRNA (SEQ ID NO: 1 ACCGUAAGUGGCGCACGGAAUU). As discussed in greater detail in the experimental section below, methylation of the genes encoding 124a miRNA has been shown for the first time to influence expression levels of the miRNA with functional consequences in terms of recognised oncogene activity. 124a miRNA is encoded by genes found at three separate loci. Thus, the methods of the invention may be carried out to determine methylation of the gene at any one, two or all of these loci. Accordingly methylation levels at the genomic loci 8p23.1/8q12.3/20q13.33 may be determined in one embodiment. As discussed above, preferably methylation status of the CpG islands identified in these genes is determined.

It is noted that the methylation status of additional genes, in particular tumour suppressor genes, may also be determined in order to supplement the methods of the invention.

In one preferred embodiment of these aspects of the invention the methylation status of the at least one gene encoding suitable miRNA, in particular the at least one genes encoding 124a miRNA, (or portion thereof, especially the CpG islands) is determined using methylation specific PCR (MSP). However, any suitable technique may be utilised to determine the methylation status of the at least one gene encoding an miRNA, which is most preferably 124a miRNA. Various methylation assay procedures are known in the art, and can be used in conjunction with the present invention. These assays rely upon two distinct approaches: bisulphite conversion based approaches and non-bisulphite based approaches. Non-bisulphite based methods for analysis of DNA methylation rely on the inability of methylation-sensitive enzymes to cleave methylation cytosines in their restriction. The bisulphite conversion relies on treatment of DNA samples with sodium bisulphite which converts unmethylated cytosine to uracil, while methylated cytosines are maintained (Furuichi et al., 1970). This conversion results in a change in the sequence of the original DNA.

DNA methylation analysis has been performed successfully with a number of techniques including: sequencing, methylation-specific PCR (MS-PCR), melting curve methylation-specific PCR (McMS-PCR), MLPA with or without bisulfite treatment, QAMA (Zeschnigk et al, 2004), MSRE-PCR (Melnikov et al, 2005), MethyLight (Eads et al., 2000), ConLight-MSP (Rand et al., 2002), bisulfite conversion-specific methylation-specific PCR (BS-MSP)(Sasaki et al., 2003), COBRA (which relies upon use of restriction enzymes to reveal methylation dependent sequence differences in PCR products of sodium bisulfite-treated DNA), methylation-sensitive single-nucleotide primer extension conformation (MS-SNuPE), methylation-sensitive single-strand conformation analysis (MS-SSCA), Melting curve combined bisulfite restriction analysis (McCOBRA)(Akey et al., 2002), PyroMethA, HeavyMethyl (Cottrell et al. 2004), MALDI-TOF, MassARRAY, Quantitative analysis of methylated alleles (QAMA), enzymatic regional methylation assay (ERMA), QBSUPT, MethylQuant, Quantitative PCR sequencing and oligonucleotide-based microarray systems, Pyrosequencing, Meth-DOP-PCR. A review of some useful techniques is provided in Nucleic acids research, 1998, Vol. 26, No. 10, 2255-2264, Nature Reviews, 2003, Vol. 3, 253_{—}266; Oral Oncology, 2006, Vol. 42, 5-13, which references are incorporated herein in their entirety. Any of these techniques may be utilised in accordance with the present invention, as appropriate.

Additional methods for the identification of methylated CpG dinucleotides utilize the ability of the methyl binding domain (MBD) of the MeCP2 protein to selectively bind to methylated DNA sequences (Cross et al, 1994; Shiraishi et al, 1999). Alternatively, the MBD may be obtained from MBP, MBP2, MBP4 or poly-MBD (Jorgensen et al., 2006). In one method, restriction exonuclease digested genomic DNA is loaded onto expressed His-tagged methyl-CpG binding domain that is immobilized to a solid matrix and used for preparative column chromatography to isolate highly methylated DNA sequences. Such methylated DNA enrichment-step may supplement the methods of the invention. Several other methods for detecting methylated CpG islands are well known in the art and include amongst others methylated-CpG island recovery assay (MIRA). Any of these methods may be employed in the present invention where desired.

The MSP technique will be familiar to one of skill in the art. In the MSP approach, DNA may be amplified using primer pairs designed to distinguish methylated from unmethylated DNA by taking advantage of sequence differences as a result of sodium-bisulfite treatment (Herman J G, Graff J R, Myohanen S, Nelkin B D, Baylin S B. Methylation-specific PCR: a novel PCR assay for methylation status of CpG islands. Proc Natl Acad Sci USA 1996; 93(18):9821-9826 and see WO 97/46705, incorporated herein by reference). After sodium-bisulfite treatment unmethylated cytosines are converted to uracil whereas methylated cytosines remain unconverted.

A specific example of the MSP technique is designated real-time quantitative MSP (QMSP), which permits reliable quantification of methylated DNA in real time. These methods are generally based on the continuous optical monitoring of an amplification procedure and utilise fluorescently labelled reagents whose incorporation in a product can be quantified and whose quantification is indicative of copy number of that sequence in the template. One such reagent is a fluorescent dye, called SYBR Green I that preferentially binds double-stranded DNA and whose fluorescence is greatly enhanced by binding of double-stranded DNA. Alternatively, labelled primers and/or labelled probes can be used. They represent a specific application of the well known and commercially available real-time amplification techniques such as hydrolytic probes (TAQMAN®), hairpin probes (MOLECULAR BEACONS®), hairpin primers (AMPLIFLUOR®), hairpin probes integrated into primers (SCORPION®)), oligonucleotide blockers and primers incorporating complementary sequences of DNAzymes (DzyNA®), specific interaction between two modified nucleotides (Plexor™) etc as described in more detail herein. Often, these real-time methods are used with the polymerase chain reaction (PCR). In Heavymethyl, the priming is methylation specific, but non-extendable oligonucleotide blockers provide this specificity instead of the primers themselves. The blockers bind to bisulfite-treated DNA in a methylation-specific manner, and their binding sites overlap the primer binding sites. When the blocker is bound, the primer cannot bind and therefore the amplicon is not generated. Heavymethyl can be used in combination with real-time detection in the methods of the invention.

Thus, in a preferred embodiment, the methylation status of the at least one gene encoding a suitable miRNA, such as 124a miRNA, is determined by methylation specific PCR/amplification, preferably real-time methylation specific PCR/amplification. In specific embodiments, the real time PCR/amplification involves use of hairpin primers (Amplifluor)/hairpin probes (Molecular Beacons)/hydrolytic probes (Taqman)/FRET probe pairs (Lightcycler)/primers incorporating a hairpin probe (Scorpion)/primers incorporating complementary sequences of DNAzymes that cleave a reporter substrate included in the reaction mixture (DzyNA®))/fluorescent dyes (SYBR Green etc.)/oligonucleotide blockers/the specific interaction between two modified nucleotides (Plexor).

Real-Time PCR detects the accumulation of amplicon during the reaction. Real-time methods do not need to be utilised, however. Many applications do not require quantification and Real-Time PCR is used principally as a tool to obtain convenient results presentation and storage, and at the same time to avoid post-PCR handling. Analyses can be performed only to know if the target DNA is present in the sample or not. End point verification is carried out after the amplification reaction has finished. This knowledge can be used in a medical diagnostic laboratory to detect a predisposition to, or the incidence of, cancer in a patient. In the majority of such cases, the quantification of DNA template is not very important. Amplification products may simply be run on a suitable gel, such as an agarose gel, to determine if the expected sized products are present. This may involve use of ethidium bromide staining and visualisation of the DNA bands under a UV illuminator for example. Alternatively, fluorescence or energy transfer can be measured to determine the presence of the methylated DNA. The end-point PCR fluorescence detection technique can use the same approaches as widely used for Real Time PCR: TaqMan assay, Molecular Beacons, Scorpion, Amplifluor etc as discussed herein. For example, <<Gene>> detector allows the measurement of fluorescence directly in PCR tubes.

In real-time embodiments, quantitation may be on an absolute basis, or may be relative to a constitutively methylated DNA standard, or may be relative to an unmethylated DNA standard. Methylation status may be determined by using the ratio between the signal of the marker under investigation and the signal of a reference gene where methylation status is known (such as β-actin for example), or by using the ratio between the methylated marker and the sum of the methylated and the non-methylated marker. Alternatively, absolute copy number of the methylated marker gene can be determined. Suitable reference genes for the present invention include beta-actin, glyceraldehyde-3-phosphate dehydrogenase (GAPDH), ribosomal RNA genes such as 18S ribosomal RNA and RNA polymerase II gene (Radonic A. et al., Biochem Biophys Res Commun. 2004 Jan. 23; 313(4):856-62). In a particularly preferred embodiment, the reference gene is beta-actin.

In one embodiment, each clinical sample is measured in duplicate and for both Ct values (cycles at which the amplification curves crossed the threshold value, set automatically by the relevant software) copy numbers are calculated. The average of both copy numbers (for each gene) is used for the result classification. To quantify the final results for each sample two standard curves are used, one for either the reference gene (β-actin or the non-methylated marker) and one for the methylated version of the marker. The results of all clinical samples (when m-Gene was detectable) are expressed as 1000 times the ratio of “copies m-Gene”/“copies β-actin” or “copies m-Gene”/“copies u-Gene+m-Gene” and then classified accordingly (methylated, non-methylated or invalid) (u=unmethylated; m=methylated).

In one embodiment, primers useful in MSP carried out on the promoter region of the respective 124a miRNA genes are provided (MIRN124A1, MIRN124A2 and MIRN124A3). These primers comprise, consist essentially of or consist of any suitable combinations selected from the following sequences:

| | |
|---|---|
| Genomic locus 1-8p23.1 | |
| miR-124a1 Methylated Sense | |
| AAAGAGTTTTTGGAAGACGTC | (SEQ ID NO: 2) |
| miR-124a1 Methylated Antisense | |
| AATAAAAAACGACGCGTATA | (SEQ ID NO: 3) |
| miR-124a1 Unmethylated Sense | |
| AATAAAGAGTTTTTGGAAGATGTT | (SEQ ID NO: 4) |
| miR-124a1 Unmethylated Antisense | |
| AAAAAAATAAAAAACAACACATATAC | (SEQ ID NO: 5) |
| Genomic locus 2-8q12.3 | |
| miR-124a2 Methylated Sense | |
| GGGTAATTAATTTGGATTTACGTC | (SEQ ID NO: 6) |
| miR-124a2 Methylated Antisense | |
| ACCGCTATTAATTAATCTATTCCG | (SEQ ID NO: 7) |
| miR-124a2 Unmethylated Sense | |
| GGGGTAATTAATTTGGATTTATGTT | (SEQ ID NO: 8) |
| miR-124a2 Unmethylated Antisense | |
| AAAACCACTATTAATTAATCTATTCCA | (SEQ ID NO: 9) |
| Genomic locus 3-20q13.33 | |
| miR-124a3 Methylated Sense | |
| GCGAGGATTTTACGTAAGTTC | (SEQ ID NO: 10) |
| miR-124a3 Methylated Antisense | |
| CCGCGTACCTTAATTATATAA | (SEQ ID NO: 11) |
| miR-124a3 Unmethylated Sense | |
| GGGTGAGGATTTTATGTAAGTTT | (SEQ ID NO: 12) |
| miR-124a3 Unmethylated Antisense | |
| TTCACCACATACCTTAATTATATAAAC | (SEQ ID NO: 13) |

Within the scope of the invention are functional derivatives of these primers. The primers may contain minor variations, such as single nucleotide substitutions, small additions and deletions, provided that their ability to act as MSP primers is not compromised. Thus, variant primers must retain the ability to distinguish between methylated and unmethylated nucleic acid molecules, in particular genes encoding 124a miRNA, following bisulphite treatment. Thus, the primers may be between 10 and 50 nucleotides in length, preferably between 12 and 40 nucleotides and most preferably between 15 and 30 nucleotides. Other variants of these sequences may be utilised in the present invention. In particular, additional sequence specific flanking sequences may be added, for example to improve binding specificity, as required. Variant sequences preferably have at least 90%, at least 91%, at least 92%, at least 93%, at least 94%, at least 95%, at least 96%, at least 97%, at least 98%, or at least 99% nucleotide sequence identity with the primers set forth herein. The primers may be adapted to allow real-time or end point fluorescence detection. For example, they may incorporate hairpin probe structures. The primers and/or hairpin structures may incorporate synthetic nucleotide analogues as appropriate or may be RNA or PNA based for example, or mixtures thereof. Similarly alternative fluorescent donor and acceptor moieties/FRET pairs may be utilised as appropriate. In addition to being labelled with the fluorescent donor and acceptor moieties, the primers may include modified oligonucleotides and other appending groups and labels provided that the functionality as a primer in the methods of the invention is not compromised. Similarly alternative fluorescent donor and acceptor moieties/FRET pairs may be utilised as appropriate. Molecules that are commonly used in real-time and end point detection techniques include fluorescein, 5-carboxyfluorescein (FAM), 2′7′-dimethoxy-4′5′-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′-dimethylaminophenylazo) benzoic acid (DABCYL), and 5-(2′-aminoethyl) aminonaphthalene-1-sulfonic acid (EDANS). Whether a fluorophore is a donor or an acceptor is defined by its excitation and emission spectra, and the fluorophore with which it is paired. For example, FAM is most efficiently excited by light with a wavelength of 488 nm, and emits light with a spectrum of 500 to 650 nm, and an emission maximum of 525 nm. FAM is a suitable donor fluorophore for use with JOE, TAMRA, and ROX (all of which have their excitation maximum at 514 nm).

Thus, in one embodiment, said donor moiety and said acceptor moiety are selected from 5-carboxyfluorescein (FAM), 2′7′-dimethoxy-4′5′-dichloro-6-carboxyfluorescein (JOE), rhodamine, 6-carboxyrhodamine (R6G), N,N,N′-tetramethyl-6-carboxyrhodamine (TAMRA), 6-carboxy-X-rhodamine (ROX), 5-(2′-aminoethyl)aminonaphthalene-1-sulfonic acid (EDANS), anthranilamide, coumarin, terbium chelate derivatives, Malachite green, Reactive Red 4, DABCYL, tetramethyl rhodamine, pyrene butyrate, eosine nitrotyrosine, ethidium, and Texas Red. In a further embodiment, said donor moiety is selected from fluorescein, 5-carboxyfluorescein (FAM), rhodamine, 5-(2′-aminoethyl)aminonaphthalene-1-sulfonic acid (EDANS), anthranilamide, coumarin, terbium chelate derivatives, Malachite green, and Reactive Red 4, and said acceptor moiety is selected from DABCYL, rhodamine, tetramethyl rhodamine, pyrene butyrate, eosine nitrotyrosine, ethidium, and Texas Red.

In one specific embodiment, said donor moiety is fluorescein or a derivative thereof, and said acceptor moiety is DABCYL. Preferably, the fluorescein derivative comprises, consists essentially of or consists of 6-carboxy fluorescein.

For all aspects and embodiments of the invention, the primers and in particular the stem loop/hairpin structures, may be labelled with donor and acceptor moieties during chemical synthesis of the primers or the label may be attached following synthesis using any suitable method. Many such methods are available and well characterised in the art.

In a further embodiment, bisulphite sequencing is utilised in order to determine the methylation status of the at least one gene encoding an miRNA, which is most preferably 124a miRNA. Primers may be designed for use in sequencing through the important CpG islands in the at least one miRNA gene. Thus, primers may be designed in both the sense and antisense orientation to direct sequencing across the promoter region of the relevant gene. In one embodiment, in which the at least one genes encoding 124a miRNA are sequenced, bisulphite sequencing may be carried out by using sequencing primers which comprise, consist essentially of or consist of the following sequences, and which may be used in isolation or in combination to sequence both strands:

| | |
|---|---|
| Genomic locus 1-8p23.1 | |
| miR-124a1 Sense | |
| AAGGATGGGGGAGAATAAAGAGTTT | (SEQ ID NO: 14) |
| miR-124a1 Antisense | |
| CTCAACCAACCCCATTCTTAACATT | (SEQ ID NO: 15) |
| Genomic locus 2-8q12.3 | |
| miR-124a2 Sense | |
| GGTAATGGTTATGAYGGAGAATATGT | (SEQ ID NO: 16) |
| miR-124a2 Antisense | |
| CCAACTCCTATCTCTACTCATCTC | (SEQ ID NO: 17) |
| Genomic locus 3-20q13.33 | |
| miR-124a3 Sense | |
| GGAAAGGGGAGAAGTGTGGGTTTT | (SEQ ID NO: 18) |
| miR-124a3 Antisense | |
| RAAAACRCCTCTCTTAACATTCACC | (SEQ ID NO: 19) |

These sequencing primers form a further aspect of the invention. Within the scope of the invention are functional derivatives of these primers. The primers may contain minor variations, such as single nucleotide substitutions, small additions and deletions, provided that their ability to act as primers in bisulphite sequencing is not compromised. Thus, variant primers must retain the ability to distinguish between methylated and unmethylated nucleic acid molecules, in particular genes encoding 124a miRNA, when used in bisulphite sequencing. The variants discussed in respect of MSP primers above applies mutatis mutandis to this aspect of the invention.

Other nucleic acid amplification techniques, in addition to PCR (which includes real-time versions thereof and variants such as nested PCR), may also be utilised, as appropriate, to detect the methylation status of the at least one gene encoding an miRNA, which is most preferably 124a miRNA. Such amplification techniques are well known in the art, and include methods such as NASBA (Compton, 1991, 3SR (Fahy et al., 1991) and Transcription Mediated Amplification (TMA). Other suitable amplification methods include the ligase chain reaction (LCR) (Barringer et al, 1990), selective amplification of target polynucleotide sequences (U.S. Pat. No. 6,410,276), consensus sequence primed polymerase chain reaction (U.S. Pat. No. 4,437,975), arbitrarily primed polymerase chain reaction (WO 90/06995) and nick displacement amplification (WO 2004/067726). This list is not intended to be exhaustive; any nucleic acid amplification technique may be used provided the appropriate nucleic acid product is specifically amplified. Thus, these amplification techniques may be tied in to MSP and/or bisulphite sequencing techniques for example.

Sequence variation that reflects the methylation status at CpG dinucleotides in the original genomic DNA offers two approaches to primer design. Firstly, primers may be designed that themselves do not cover any potential sites of DNA methylation. Sequence variation at sites of differential methylation are located between the two primers. Such primers are used in bisulphite genomic sequencing, COBRA and Ms-SnuPE for example. Secondly, primers may be designed that anneal specifically with either the methylated or unmethylated version of the converted sequence. If there is a sufficient region of complementarity, e.g., 12, 15, 18, or 20 nucleotides, to the target, then the primer may also contain additional nucleotide residues that do not interfere with hybridization but may be useful for other manipulations. Examples of such other residues may be sites for restriction endonuclease cleavage, for ligand binding or for factor binding or linkers or repeats. The oligonucleotide primers may or may not be such that they are specific for modified methylated residues.

One way to distinguish between modified and unmodified DNA is to hybridize oligonucleotide primers which specifically bind to one form or the other of the DNA. After hybridization, an amplification reaction can be performed and amplification products assayed. The presence of an amplification product indicates that a sample hybridized to the primer. The specificity of the primer indicates whether the DNA had been modified or not, which in turn indicates whether the DNA had been methylated or not.

Another related means for distinguishing between modified and unmodified DNA is to use oligonucleotide probes which may also be specific for certain products. Such probes may be hybridized directly to modified DNA or to amplification products of modified DNA. Oligonucleotide probes can be labelled using any detection system known in the art. These include but are not limited to fluorescent moieties, radioisotope labelled moieties, bioluminescent moieties, luminescent moieties, chemiluminescent moieties, enzymes, substrates, receptors, or ligands.

In the MSP technique, amplification is achieved with the use of primers specific for the sequence of the gene whose methylation status is to be assessed. In order to provide specificity for the nucleic acid molecules, primer binding sites corresponding to a suitable region of the sequence may be selected. The skilled reader will appreciate that the nucleic acid molecules may also include sequences other than primer binding sites which are required for detection of the methylation status of the gene, for example RNA Polymerase binding sites or promoter sequences may be required for isothermal amplification technologies, such as NASBA, 3SR and TMA.

TMA (Gen-probe Inc.) is an RNA transcription amplification system using two enzymes to drive the reaction, namely RNA polymerase and reverse transcriptase. The TMA reaction is isothermal and can amplify either DNA or RNA to produce RNA amplified end products. TMA may be combined with Gen-probe's Hybridization Protection Assay (HPA) detection technique to allow detection of products in a single tube. Such single tube detection is a preferred method for carrying out the invention.

As mentioned above, in a preferred embodiment, the methylation status of the at least one gene encoding an miRNA, which is most preferably 124a miRNA, is determined by methylation specific PCR, preferably real-time or end-point methylation specific PCR.

Since the promoters of the genes encoding 124a miRNA appear to be almost completely unmethylated in normal tissues, and highly methylated in cancer, it is clear that the methods of the invention are particularly useful, since the detection of methylation in this region is readily observable as being significant in terms of a cancer diagnosis and indeed in diagnosis of any other disease in which 124a miRNA is involved and also in selecting suitable treatment regimens and for determining the likelihood of successful treatment or resistance to treatment with certain anti-cancer agents. Likewise the detection of unmethylated genes encoding 124a miRNA is also of relevance.

However, when determining methylation status, it may still be beneficial to include suitable controls in order to ensure the method chosen to assess this parameter is working correctly and reliably. For example, suitable controls may include assessing the methylation status of a gene known to be methylated. This experiment acts as a positive control to help to ensure that false negative results are not obtained (i.e. a conclusion of a lack of methylation is made even though the at least one gene encoding an miRNA, which is most preferably 124a miRNA may, in fact, be methylated). The gene may be one which is known to be methylated in the sample under investigation or it may have been artificially methylated, for example by using a suitable methyltransferase enzyme, such as Sssl methyltransferase. In one embodiment, the at least one gene encoding an miRNA, which is most preferably 124a miRNA, may be assessed in normal cells such as colon cells, following treatment with Sssl methyltransferase, as a positive control. Alternatively, the positive control may be a suitable diseased sample in which the gene is known to be methylated.

Additionally or alternatively, suitable negative controls may be employed with the methods of the invention. Here, suitable controls may include assessing the methylation status of a gene known to be unmethylated. This experiment acts as a negative control and helps to ensure that false positive results are not obtained (i.e. a conclusion of methylation is made even though the at least one gene encoding an miRNA, which is most preferably 124a miRNA may, in fact, be unmethylated). The gene may be one which is known to be unmethylated in the sample under investigation or it may have been artificially demethylated, for example by using a suitable DNA methyltransferase inhibitor, such as those discussed in more detail herein. In one embodiment, the at least one gene encoding an miRNA, which is most preferably 124a miRNA, may be assessed in normal cells such as colon cells as a negative control, since it has been shown for the first time herein that the 124a miRNA gene is almost completely unmethylated in normal tissues.

The level of methylation of at least one gene encoding an miRNA, which is most preferably 124a miRNA may, as necessary, be measured in order to determine if it is statistically significant in the sample. This helps to provide a reliable test for the methods of the invention. Any method for determining whether the levels of methylation of at least one gene encoding an miRNA, which is most preferably 124a miRNA is significant may be utilised. Such methods are well known in the art and routinely employed. For example, statistical analyses may be performed using an analysis of variance test. Typical P values for use in such a method would be P values of <0.05 or 0.01 or 0.001 when determining whether the relative levels of methylation are statistically significant. A change in methylation, as compared to a suitable negative control as defined above, may be deemed significant if there is at least a 10% increase in methylation for example. The test may be made more selective by making the change at least 15%, 20%, 25%, 30%, 35%, 40% or 50%, for example, in order to be considered statistically significant.

In a particularly preferred embodiment of the invention, the disease which is associated with methylation of at least one gene encoding an miRNA, which is most preferably 124a miRNA is cancer. Preferably, the disease or cancer is not blastoma, in particular neuroblastoma, or sarcoma. In a particularly preferred embodiment of the invention, the cancer is selected from colon cancer, breast cancer, lung cancer, leukaemia and lymphoma. In all of these disease conditions, methylation of the gene encoding 124a miRNA and consequential reduced expression of 124a miRNA has been shown to be diagnostically relevant. A most preferred disease condition in which methylation of miRNA genes is relevant is colon cancer.

### Expression Level Methods

The 124a miRNA is the first miRNA shown to be differentially methylated between a disease state and a non-disease state. In particular, loss of 124a miRNA expression has been shown for the first time herein to be linked to the incidence of cancer.

Accordingly, in a further aspect, the invention provides a method of diagnosing a disease associated with a loss of 124a miRNA expression comprising, in a test sample obtained from a subject, determining the expression levels or activity of 124a miRNA in the sample, wherein a reduced level of expression or activity is indicative of the presence of the disease.

In a related aspect, the invention also provides a method for predicting the likelihood of successful treatment of a disease associated with loss of 124a miRNA expression with a DNA demethylating agent and/or a DNA methyltransferase inhibitor and/or a HDAC inhibitor comprising determining the expression levels or activity of 124a miRNA in a test sample obtained from a subject, wherein a reduced level of expression or activity is indicative of the likelihood of successful treatment being higher than if the expression levels or activity are higher.

The opposite scenario is also envisaged in the present invention. Thus, in a related aspect, the invention provides a method for predicting the likelihood of resistance to treatment of a disease associated with loss of 124a miRNA expression with a DNA demethylating agent and/or a DNA methyltransferase inhibitor and/or a HDAC inhibitor comprising determining the expression levels or activity of 124a miRNA in a test sample obtained from a subject, wherein an increased level of expression or activity is indicative of the likelihood of successful treatment being lower than if the expression levels or activity are lower.

Furthermore, the invention also relates to a method of selecting a suitable treatment regimen for a disease associated with loss of 124a miRNA expression comprising determining the expression levels or activity of 124a miRNA in a test sample obtained from a subject, wherein if the expression levels or activity are lower/reduced a DNA demethylating agent and/or a DNA methyltransferase inhibitor and/or a HDAC inhibitor is selected for treatment.

The opposite scenario is also envisaged in the present invention. Thus, in a related aspect if the expression levels or activity of 124a miRNA are higher/increased, treatment using a DNA demethylating agent and/or a DNA methyltransferase inhibitor and/or a HDAC inhibitor is contra-indicated.

Confirmation of the effect of the DNA demethylating agent and/or a DNA methyltransferase inhibitor and/or a HDAC inhibitor may be sought by further contacting the sample with the DNA demethylating agent and/or a DNA methyltransferase inhibitor and/or a HDAC inhibitor and determining whether the expression level of 124a miRNA is altered.

These methods may be combined with the methods described above including determining the methylation status of miRNA encoding genes, as appropriate.

For the avoidance of doubt, it is confirmed that all relevant definitions and embodiments provided above in respect of methods including determination of the methylation status of miRNA encoding genes apply mutatis mutandis to the aspects of the invention in which expression of 124a miRNA is monitored.

The level of expression of 124a miRNA may, as necessary, be measured in order to determine if it is statistically significant in the sample. This helps to provide a reliable test for the methods of the invention. Any method for determining whether the expression level of 124a miRNA is significantly reduced may be utilised. Such methods are well known in the art and routinely employed. For example, statistical analyses may be performed using an analysis of variance test. Typical P values for use in such a method would be P values of <0.05 or 0.01 or 0.001 when determining whether the relative expression or activity is statistically significant. A change in expression may be deemed significant if there is at least a 10% decrease for example. The test may be made more selective by making the change at least 15%, 20%, 25%, 30%, 35%, 40% or 50%, for example, in order to be considered statistically significant.

In a preferred embodiment, the decreased level of expression of 124a miRNA is determined with reference to a control sample. This control sample is preferably taken from normal (i.e. non tumorigenic) tissue in the subject, where expression of 124a miRNA is normal. Additionally or alternatively control samples may also be utilised in which there is known to be a lack of expression of 124a miRNA.

Suitable additional controls may also be included to ensure that the test is working properly, such as measuring levels of expression or activity of a suitable reference gene in both test and control samples.

In a most preferred embodiment, the subject is a human subject. Generally, the subject will be a patient wherein cancer is suspected or a potential cancer has been identified and the method may be used to determine if indeed there is a cancer present. The methods of the invention may be used in conjunction with known methods for detecting cancer.

A decreased or abolished level of 124a miRNA expression, caused by methylation of the gene, results in lower levels of functional 124a miRNA and this is indicative of cancer and/or likelihood of successful treatment and/or directs the course of treatment. Assessment of expression levels of 124a miRNA may be monitored at the primary transcript, precursor or mature miRNA level. Thus, miRNA genes are generally transcribed in the nucleus by RNA polymerase II to form long primary transcripts (pri-miRNAs), which are capped with 7-methyl-guanosine and are also polyadenylated. Pri-miRNAs are then processed into approximately 60 nucleotide precursor miRNAs (pre-miRNAs) which may form imperfect stem-loop structures by virtue of the activity of Drosha (a nuclear RNase III enzyme) and its co-factor Pasha. Pre-miRNAs are transported into the cytoplasm and subsequently cleaved by the cytoplasmic RNase III enzyme Dicer into mature miRNAs.

Suitable methods for determining expression of 124a miRNA at the RNA level are well known in the art. Methods employing nucleic acid probe hybridization to the relevant 124a miRNA transcript may be employed for measuring the presence and/or level of 124a miRNA. Such methods include use of nucleic acid probe arrays (microarray technology) and Northern blots. Advances in genomic technologies now permit the simultaneous analysis of thousands of genes, although many are based on the same concept of specific probe-target hybridization.

Sequencing-based methods are an alternative. These methods started with the use of expressed sequence tags (ESTs), and now include methods based on short tags, such as serial analysis of gene expression (SAGE) and massively parallel signature sequencing (MPSS). Differential display techniques provide yet another means of analyzing gene expression; this family of techniques is based on random amplification of cDNA fragments generated by restriction digestion, and bands that differ between two tissues identify cDNAs of interest.

In one embodiment, the levels of 124a miRNA expression are determined using reverse transcriptase polymerase chain reaction (RT-PCR). RT-PCR is a well known technique in the art which relies upon the enzyme reverse transcriptase to reverse transcribe mRNA to form cDNA, which can then be amplified in a standard PCR reaction. Protocols and kits for carrying out RT-PCR are extremely well known to those of skill in the art and are commercially available.

In a specific embodiment, RT-PCR determination of miRNA expression includes use of primers comprising, consisting essentially of or consisting of the following nucleotide sequences:

(SEQ ID NO: 32)

5′-GTT CAC AGC GGA CCT TGA TT-3′ (sense)
and

(SEQ ID NO: 33)

5′-ACC GCG TGC CTT AAT TGT AT-3′ (antisense) for
miR-124a.

Functional derivatives of these sequences are also envisaged. The primers may contain minor variations, such as single nucleotide substitutions, small additions and deletions, provided that their ability to act as RT-PCR primers is not compromised. Thus, variant primers must retain the ability to allow determination of expression of 124a miRNA through RT-PCR techniques. Thus, the primers may be between 10 and 50 nucleotides in length, preferably between 12 and 40 nucleotides and most preferably between 15 and 30 nucleotides.

In a preferred embodiment, the RT-PCR is carried out in real time and in a quantitative manner. Real time quantitative RT-PCR has been thoroughly described in the literature (see Gibson et al for an early example of the technique) and a variety of techniques are possible. End point detection may also be utilised as desired, as discussed herein. Examples include use of hydrolytic probes (TAQMAN®), hairpin probes (MOLECULAR BEACONS®), hairpin primers (AMPLIFLUOR®), hairpin probes integrated into primers (SCORPION®), oligonucleotide blockers and primers incorporating complementary sequences of DNAzymes (DzyNA®), specific interaction between two modified nucleotides (Plexor™) etc as described in more detail herein. Often, these real-time methods are used with the polymerase chain reaction (PCR). All of these systems are commercially available and well characterised, and may allow multiplexing (that is, the determination of expression of multiple genes in a single sample).

These techniques produce a fluorescent read-out that can be continuously monitored. Real-time techniques are advantageous because they keep the reaction in a “single tube”. This means there is no need for downstream analysis in order to obtain results, leading to more rapidly obtained results. Furthermore, keeping the reaction in a “single tube” environment reduces the risk of cross contamination and allows a quantitative output from the methods of the invention. This may be particularly important in the clinical setting of the present invention.

In one specific embodiment, real-time RT-PCR is carried out using SYBR Green as the detection reagent. This reagent is commercially available as a PCR master mix from Applied Biosystems. In one specific embodiment, real time RT-PCR includes use of an antisense oligonucleotide comprising, consisting essentially of or consisting of the nucleotide sequence GCGAGCACAGAATTAATACGACTC (SEQ ID NO: 37), optionally together with a sense primer comprising, consisting essentially of or consisting of the nucleotide sequence TTA AGG CAC GCG GTG MT GCC A (miR-124a) (SEQ ID NO: 38). Again, functional derivatives which retain function in the real-time RT-PCR assay are envisaged within the scope of the invention (as defined above).

It should be noted that whilst PCR is a preferred amplification method, to include variants on the basic technique such as nested PCR, equivalents may also be included within the scope of the invention. Examples include isothermal amplification techniques such as NASBA, 3SR, TMA and triamplification, all of which are well known in the art and commercially available. Other suitable amplification methods include the ligase chain reaction (LCR) (Barringer et al, 1990), selective amplification of target polynucleotide sequences (U.S. Pat. No. 6,410,276), consensus sequence primed polymerase chain reaction (U.S. Pat. No. 4,437,975), arbitrarily primed polymerase chain reaction (WO90/06995) and nick displacement amplification (WO2004/067726).

In one embodiment, the methods of the invention are carried out by determining protein expression of CDK6 as an indication of the methylation status/expression level of the miRNA. Thus, methylation of the genes encoding 124a miRNA has been shown herein to cause down regulation of expression of 124a miRNA and this leads to, as a direct functional consequence, an increase in the translation of the oncogene CDK6. This gene was identified and confirmed as a target of 124a miRNA herein for the first time, as described in further detail in the experimental section below. In a preferred embodiment, high levels of protein expression of CDK6 is observed in the sample in order to conclude a diagnosis of cancer, or to make a decision on the best course of treatment in accordance with the other methods of the invention. High levels of expression are indicative of methylation of 124a miRNA and thus reduced levels of 124a miRNA.

Relative levels of expression may be determined against suitable controls, as discussed herein in further detail. Levels of protein expression may be determined by a number of techniques, as are well known to one of skill in the art. Examples include western blots, immunohistochemical staining and immunolocalization, immunofluorescene, enzyme-linked immunosorbent assay (ELISA), immunoprecipitation assays, agglutination reactions, radioimmunoassay, flow cytometry and equilibrium dialysis. These methods generally depend upon a reagent specific for identification of CDK6. The reagent is preferably an antibody and may comprise monoclonal or polyclonal antibodies. Fragments and derivatized (such as humanized) antibodies may also be utilised, to include without limitation Fab fragments, ScFv, single domain antibodies, nanobodies, heavy chain antibodies etc which retain specific CDK6 binding function. Any detection method may be employed in accordance with the invention. The nature of the reagent is not limited except that it must be capable of specifically identifying CDK6. Additional examples include lectins, receptors and nucleic acid based reagents (DNA/RNA etc.) Of course, in the case of a positive diagnosis of cancer, there will be high levels of CDK6. This method is favourable, since a positive result is investigated rather than a negative one. The level of expression of CDK6 may, as necessary, be measured in order to determine if it is statistically significant in the sample. This helps to provide a reliable test for the methods of the invention. Any method for determining whether the expression level of CDK6 is significantly increased may be utilised. Such methods are well known in the art and routinely employed. For example, statistical analyses may be performed using an analysis of variance test. Typical P values for use in such a method would be P values of <0.05 or 0.01 or 0.001 when determining whether the relative expression or activity is statistically significant. A change in expression may be deemed significant if there is at least a 10% increase for example. The test may be made more selective by making the change at least 15%, 20%, 25%, 30%, 35%, 40% or 50%, for example, in order to be considered statistically significant.

Suitable controls are described above and that discussion applies here mutatis mutandis.

Also shown herein is the effect of CDK6 kinase activity on retinoblastoma (Rb) phosphorylation. Rb is a tumour suppressor gene and represents a target of CDK6 activity. Specifically, measuring levels of phosphorylation of Rb, in particular at residues 807 and 811 (based upon the wild type amino acid sequence) provides an indication of the activity of CDK6 which in turn may provide an insight into the methylation status of the genes encoding miR-124a and expression levels of 124a miRNA. Thus, assessing phosphorylation of Rb can also be useful in the present invention. Methods of determining levels of protein phosphorylation are known in the art and include use of detection reagents such as antibodies (including derivatives that retain specific binding affinity) specific for the phosphorylated form of the protein. Examples include use of Western Blots, Immunohistochemistry etc. The discussions of determining the significance in changes to phosphorylation levels, including comparison with suitable controls, may be derived with reference to the more detailed discussion above, which applies here mutatis mutandis.

In similar fashion, the description of preferred disease types, sample types, subjects etc. above in respect of “methylation” related aspects of the invention apply mutatis mutandis to the “expression” aspects of the invention. Thus preferably, the cancer is selected from colon cancer, breast cancer, lung cancer, leukaemia and lymphoma. The cancer is most preferably colon cancer. Also, as aforementioned, the 124a miRNA may be one encoded by the gene or genes found the genomic loci 8p23.1 (124a1 miRNA)/8q12.3 (124a2 miRNA)/20q13.33 (124a3 miRNA).

In further embodiments of both the “methylation” and “expression” aspects of the invention, which may be additional to the basic methods or utilised as alternatives, acetylation of histones H3/H4 is used as an indication of the methylation status of the at least one gene encoding expression level of the miRNA. As is shown in the experimental section below, acetylation of both histones H3 and H4 is linked to demethylation of 124a miRNA and a consequential up-regulation in expression. In cancer cells, 124a miRNA is methylated leading to lower levels of 124a miRNA and this is linked to an absence of histone H3 and H4 acetylation in the CpG island of the 124a miRNA gene.

In an additional or alternative embodiment, the methods of the invention incorporate determining the methylation status of histone H3 as an indication of the methylation status of the gene encoding/expression level of the miRNA. As is shown in the experimental section below, methylation of histone H3 is linked to methylation of 124a miRNA encoding genes and a consequential down-regulation in 124a miRNA expression in cancer cells. Removal of methylation of the one or more genes encoding 124a miRNA leading to increased levels of 124a miRNA is linked to a loss of histone H3 methylation. In a specific embodiment, trimethylation of lysine 4 of histone H3 is measured as an indication of the methylation status of the miRNA encoding gene.

In a related embodiment, occupancy of the miRNA gene by methyl binding domain containing proteins may be utilised as an indication of the methylation status of the at least one gene encoding/expression level of the miRNA. As is shown in the experimental section below, demethylation of 124a miRNA encoding genes and a consequential up-regulation in expression is mirrored by an absence of methyl binding domain containing proteins. In cancer cells, the genes encoding 124a miRNA are methylated leading to lower levels of 124a miRNA and this is linked to the presence of methyl binding domain containing proteins in the CpG island of the 124a miRNA gene. Preferred methyl binding domain containing proteins include MeCP2 and/or MBD2.

For these methods in which the CpG island is monitored, preferably chromatin immunoprecipitation is utilised in order to determine the respective indication of the methylation status of the at least one gene encoding the miRNA. Chromatin immunoprecipitation is a well known technique in the art (see for example Ballestar, E et al. Methyl-CpG binding proteins identify novel sites of epigenetic inactivation in human cancer. EMBO J. 22, 6335-6345 (2003)) which relies upon cross-linking of the binding protein to the DNA, followed by isolation, shearing of the DNA, antibody detection and isolation by precipitation. The isolated DNA is then released from the binding protein by reversing the cross-linking and is amplified by PCR to determine where the binding protein was bound (Metivier, R. et al., Estrogen receptor-alpha directs ordered, cyclical, and combinatorial recruitment of cofactors on a natural target promoter, Cell 2003, 115(6) P751-63). However, any other suitable technique may be employed as appropriate.

In one specific embodiment, primers comprising, consisting essentially of or consisting of the following nucleotide sequences are used in the chromatin immunoprecipitation methods of the invention:

miR-124a1

(SEQ ID NO: 40)

5′-CAA AGA GCC TTT GGA AGA CG-3′ (sense)
and

(SEQ ID NO: 41)

5′-GGA AGA GGG GTG GGT AGA AG-3′ (antisense)
miR-124a2

(SEQ ID NO: 42)

5′-GCG TGG TCC TTA AAA ACC TG-3′ (sense)
and

(SEQ ID NO: 43)

5′-CCA TGC CAT TTA CAG CAC AC-3′ (antisense)
miR-124a3

(SEQ ID NO: 44)

5′-GGA GAA GTG TGG GCT CCT C-3′ (sense)
and

(SEQ ID NO: 45)

5′-AAT CAA GGT CCG CTG TGA AC-3′ (antisense).

Functional derivatives of these primers are also encompassed within the scope of the present invention. The primers may contain minor variations, such as single nucleotide substitutions, small additions and deletions, provided that their ability to act as primers in the appropriate chromatin immunoprecipitation methods is not compromised. Thus, variant primers must retain the ability to amplify the appropriate PCR product. Thus, the primers may be between 10 and 50 nucleotides in length, preferably between 12 and 40 nucleotides and most preferably between 15 and 30 nucleotides. The discussion provided in respect of variants of MSP primers applies mutatis mutandis here.

### Methods of Treatment, Compositions Etc.

In a further aspect the invention provides a method of treating a condition characterised by methylation of at least one gene encoding an miRNA comprising administering to a patient in need of treatment a demethylating agent in an amount effective to restore miRNA function/expression (by removing methylation of the one or more miRNA encoding genes). The method preferably comprises administration of a DNA demethylating agent and/or a DNA methyltransferase inhibitor and/or a HDAC inhibitor. Preferably, the subject has been selected for treatment on the basis of determining the methylation status of an miRNA encoding gene, in particular at least one 124a miRNA gene, according to the methods of the invention. In an alternative or additional embodiment, the subject is selected for treatment on the basis of measuring the expression levels of 124a miRNA according to any of the methods of the invention.

Thus, for the patient population where an miRNA encoding gene and preferably at least one gene encoding 124a miRNA is methylated, which leads to decreased gene expression, this type of treatment is recommended. Preferably, treatment involves use of a DNA demethylating agent and/or a DNA methyltransferase inhibitor and/or a HDAC inhibitor. This method is referred to hereinafter as the “method of treatment” aspect of the invention.

In a related aspect, the invention also provides for the use of a demethylating agent such as a DNA demethylating agent and/or a DNA methyltransferase inhibitor and/or a HDAC inhibitor in the manufacture of a medicament for use in treating a condition characterised by methylation of one or more genes encoding an miRNA in a subject. This aspect may also be considered as a DNA methylation agent and/or a DNA methyltransferase inhibitor and/or a HDAC inhibitor for use in treating a condition characterised by methylation of one or more genes encoding an miRNA in a subject. Preferably, the subject has been selected for treatment on the basis of determining the methylation status of a gene encoding an miRNA, in particular 124a miRNA, according to any of the (specific) methods of the invention. Additionally or alternatively, the subject may be selected for treatment on the basis of measuring the expression levels of an miRNA, in particular 124a miRNA, according to any of the methods of the invention.

Thus, the patient population may be selected for treatment on the basis of their methylation status with respect to the relevant gene encoding an miRNA, in particular 124a miRNA which leads to down regulation of gene expression of the corresponding miRNA. This leads to a much more focussed and personalised form of medicine and thus leads to improved success rates since patients will be treated with drugs which are most likely to be effective. In the experimental section below, it is shown for the first time that the methylation status of an miRNA gene is disease associated and may, therefore, be useful for indicating whether certain treatments such as use of DNA methyltransferase inhibitors and/or a HDAC inhibitors is likely to prove successful.

In a related aspect the invention also provides a method of treating a condition characterised by methylation of one or more genes encoding an miRNA comprising administering to a patient in need of treatment one or more miRNAs, wherein the administered miRNAs complement miRNA function suppressed by methylation in the patient. This may be considered as one or more miRNAs for use in treating a condition characterised by methylation of one or more genes encoding an miRNA, wherein the administered miRNAs complement miRNA function suppressed by methylation in the patient or as use of one or more miRNAs in the manufacture of a medicament for treating a condition characterised by methylation of one or more genes encoding an miRNA, wherein the administered miRNAs complement miRNA function suppressed by methylation in the patient.

The invention also provides a method for treating a condition characterised by methylation of one or more genes encoding an miRNA in a subject, said subject having a reduced level or activity of the one or more miRNAs, in particular 124a miRNA, comprising reconstitution of miRNA expression in the subject. This may be achieved by alleviating methylation of the relevant miRNA encoding genes or supplying suitable miRNAs to restore functionality for example, as described herein.

For the avoidance of doubt, when reference is made to methods of treatment, reference is intended to also be made to the uses described herein.

Thus, methylation of miRNA encoding genes, in particular the 124a miRNA genes, has been shown for the first time herein to be relevant to cancer. Methylation is linked to down regulation of miRNA expression. Accordingly, it is predicted that methods for increasing miRNA expression will result in improved recovery from cancer in cases where the relevant miRNA gene is methylated. As shown in the experimental section below, reconstitution of functional miRNA produces tumour suppressor like features in transfected cells.

In all aspects of the invention the most preferred miRNA is 124a miRNA. Thus, the miRNA comprises 124a miRNA having the sequence ACCGUAAGUGGCGCACGGAAUU (SEQ ID NO: 1), or a functional derivative thereof, as defined and described in greater detail herein.

In a particularly preferred embodiment of the invention, the disease which is treated is cancer. Preferably, the disease or cancer is not blastoma, in particular neuroblastoma, or sarcoma. In a particularly preferred embodiment of the invention, the cancer is selected from colon cancer, breast cancer, lung cancer, leukaemia and lymphoma. In all of these disease conditions, methylation of the gene encoding 124a miRNA and consequential reduced expression of 124a miRNA has been shown. A most preferred disease condition in which methylation of miRNA genes is relevant and in which patients may be selected and treated in accordance with the methods of the invention is colon cancer.

In methods involving reconstitution of functional miRNA activity, a recombinant construct capable of expressing an miRNA comprising the nucleotide sequence ACCGUAAGUGGCGCACGGAAUU (SEQ ID NO: 1) or a functional derivative thereof with the ability to down-regulate CDK6 expression is provided. Suitable recombinant constructs may be based upon suitable constructs known in the art and commercially available. Thus, the invention provides a recombinant construct capable of directing transcription of a nucleotide sequence to produce the 124a miRNA sequence. Variants include all those which retain functionality in terms of their ability to inhibit translation of the oncogene CDK6. Thus all functional derivatives of the 124a miRNA sequence which retain the ability to down-regulate expression of CDK6 at the protein level are included within the scope of the invention. Functional derivatives include those having nucleotide additions, substitutions and deletions and may include non-natural bases as appropriate.

Recombinant constructs according to the invention may include the full gene sequence encoding the pri-miRNA and/or pre-miRNA from any suitable source, although the nucleotide sequence is preferably that from humans. Preferred is inclusion of the genes MIRN124A1/MIRN124A2/MIRN124A3. The nucleotide sequence contained within the recombinant construct may also allow for minor variations provided that the miRNA finally expressed by the construct retains functionality in terms of its ability to inhibit CDK6 production. The nucleic acid molecule which encodes the 124a miRNA may be operably linked to a suitable regulatory sequence in order to direct specific expression in a host cell. The nature of the regulatory sequences employed will be determined at least in part by the nature of the host cell used to produce the miRNA. The term “regulatory sequence” is to be taken in a broad context and refers to a regulatory nucleic acid capable of effecting expression of the nucleic acid molecule to which it is operably linked. Encompassed by the aforementioned term are promoters and nucleic acids or synthetic fusion molecules or derivatives thereof which activate or enhance expression of a nucleic acid, so-called “activators” or “enhancers”. The term “operably linked” as used herein refers to a functional linkage between the “promoter” sequence and the nucleic acid molecule of interest, such that the “promoter” sequence is able to initiate transcription of the nucleic acid molecule to produce the 124a miRNA.

A preferred regulatory sequence is a promoter, which may be a constitutive or an inducible promoter. In a most preferred embodiment, expression is driven by at least one, preferably one, RNA polymerase promoter. Preferred promoters are inducible promoters to allow tight control of expression of the miRNA. Promoters inducible through use of an appropriate chemical, such as IPTG are preferred. Alternatively, the transgene encoding the miRNA is placed under the control of a strong constitutive promoter Preferably, any promoter which is used will direct strong expression of the miRNA. The nature of the promoter utilised may, in part, be determined by the specific host cell utilised to produce the RNA. In one embodiment, the regulatory sequence comprises a bacteriophage promoter, such as a T7, T3, SV40 or SP6 promoter, most preferably a T7 promoter. In yet other embodiments of the present invention, other promoters useful for the expression of RNA are used and include, but are not limited to, promoters from an RNA Pol I, an RNA Pol II or an RNA Pol III polymerase. Other promoters derived from yeast or viral genes may also be utilised as appropriate.

In an alternative embodiment, the regulatory sequence comprises a promoter selected from the well known tac, trc and lac promoters. Inducible promoters suitable for use with bacterial hosts include β-lactamase promoter, E. coli λ phage PL and PR promoters, and E. coli galactose promoter, arabinose promoter and alkaline phosphatase promoter. Therefore, the present invention also encompasses a method for generating 124a miRNA. This method comprises the steps of introducing (e.g. by transformation, transfection or injection) a recombinant construct of the invention into a host cell of the invention under conditions that allow transcription from said recombinant (DNA) construct to produce the miRNA.

Optionally, one or more transcription termination sequences or “terminators” may also be incorporated in the recombinant construct of the invention. The term “transcription termination sequence” encompasses a control sequence at the end of a transcriptional unit, which signals 3′ processing and poly-adenylation of a primary transcript and termination of transcription. As mentioned above, typically miRNAs are produced via pri- and pre-miRNA transcripts which are polyadenylated. The transcription termination sequence is useful to prevent read through transcription such that the miRNA is accurately produced in or by the host cell. In one embodiment, the terminator comprises a T7, T3, SV40 or SP6 terminator, preferably a T7 terminator. Other terminators derived from yeast or viral genes may also be utilised as appropriate.

Additional regulatory elements, such as transcriptional enhancers, may be incorporated in the expression construct. The recombinant constructs of the invention may further include an origin of replication which is required for maintenance and/or replication in a specific cell type. One example is when an expression construct is required to be maintained in a bacterial cell as an episomal genetic element (e.g. plasmid or cosmid molecule) in a cell. Preferred origins of replication include, but are not limited to, f1-ori and colE1 ori. The recombinant construct may optionally comprise a selectable marker gene. As used herein, the term “selectable marker gene” includes any gene, which confers a phenotype on a cell in which it is expressed to facilitate the identification and/or selection of cells, which are transfected or transformed, with a recombinant construct of the invention. Examples of suitable selectable markers include resistance genes against ampicillin (Ampr), tetracycline (Tcr), kanamycin (Kanr), phosphinothricin, and chloramphenicol (CAT) gene. Other suitable marker genes provide a metabolic trait, for example manA. Visual marker genes may also be used and include for example beta-glucuronidase (GUS), luciferase and Green Fluorescent Protein (GFP).

In addition, the invention also relates to a host cell comprising a recombinant construct of the invention. The host cell expressing the miRNA molecule may be any suitable host cell, including both prokaryotic and eukaryotic cells. According to one embodiment, any bacterium or cyanobacterium that is capable of expressing the miRNA molecule from the recombinant constructs of the invention can be used. The bacterium may be chosen from the group comprising Gram-negative and Gram-positive bacteria, such as, but not limited to, Escherichia spp. (e.g. E. coli), Pseudomonas spp., Enterobacter spp., Bacillus spp. (e.g. B. thuringiensis), Rhizobium spp., Lactobacilllus spp., Lactococcus spp., etc. Preferred bacteria include those from the genus Escherichia, in particular E. coli and all suitable strains thereof. According to another embodiment, the host cell comprises a unicellular eukaryote or an organism from the Kingdom Protista. Unicellular organism for use in the methods of the invention include, by way of example and not limitation, algae and yeast. Yeast cells represent a preferred host cell, in particular well characterised unicellular yeast such as those from the genus Saccharomyces e.g. S. cerevisiae or Schizosaccharomyces pombe. Transformation and stable expression can be achieved in yeast cells by routine methods (for example using YACS, see Sambrook and Russell—Molecular Cloning: A laboratory manual—third edition and the references provided therein in Protocol 10). A yeast recombinant construct can typically include one or more of the following: a promoter sequence, fusion partner sequence, leader sequence, transcription termination sequence, a selectable marker. These elements can be combined into an expression cassette, which may be maintained in a replicon, such as an extrachromosomal element (e.g., plasmids) capable of stable maintenance in a host, such as yeast or bacteria. The replicon may have two replication systems, thus allowing it to be maintained, for example, in yeast for expression and in a prokaryotic host for cloning and amplification. Examples of such yeast-bacteria shuttle vectors include YEp24 (Botstein et al., 1979), pCI/1 (Brake et al., 1984), and YRp17 (Stinchcomb et al., 1982). In addition, a replicon may be either a high or low copy number plasmid. A high copy number plasmid will generally have a copy number ranging from about 5 to about 200, and typically about 10 to about 150. A host containing a high copy number plasmid will preferably have at least about 10, and more preferably at least about 20. Useful yeast promoter sequences can be derived from genes encoding enzymes in the metabolic pathway. Examples of such genes include alcohol dehydrogenase (ADH) (EP 0 284044), enolase, glucokinase, glucose-6-phosphate isomerase, glyceraldehyde-3-phosphatedehydrogenase (GAP or GAPDH), hexokinase, phosphofructokinase, 3-phosphoglycerate mutase, and pyruvate kinase (PyK). The yeast PHO5 gene, encoding acid phosphatase, also provides useful promoter sequences (Myanohara et al., 1983). In addition, synthetic promoters that do not occur in nature also function as yeast promoters. Examples of such hybrid promoters include the ADH regulatory sequence linked to the GAP transcription activation region (U.S. Pat. Nos. 4,876,197 and 4,880,734). Examples of transcription terminator sequences and other yeast-recognized termination sequences, such as those coding for glycolytic enzymes, are known to those of skill in the art.

Alternatively, the expression constructs can be integrated into the yeast genome with an integrating vector. Integrating vectors typically contain at least one sequence homologous to a yeast chromosome that allows the vector to integrate, and preferably contain two homologous sequences flanking the expression construct. Integrations appear to result from recombinations between homologous DNA in the vector and the yeast chromosome (Orr-Weaver et al., 1983). An integrating vector may be directed to a specific locus in yeast by selecting the appropriate homologous sequence for inclusion in the vector (Orr-Weaver et al., 1983).

In one embodiment, the host cells are inactivated following expression of the miRNA. These host cells may then be utilised in the methods of treatment of the invention together with suitable excipients or carriers or diluents Suitable inactivation techniques include heat treatment or chemical treatment, for example through use of phenol or formaldehyde.

Other suitable host cells include those derived from animals such as CHO cells etc., which are routinely used to express products for pharmaceutical use. Examples of mammalian cells which may be used to produce miRNA include the tetracycline resistant cells commercially available from Invitrogen. The miRNA once expressed by any suitable host cell may be isolated prior to use in the methods of treatment of the invention. This may be achieved by any suitable means such as by routine RNA extraction followed by gel purification or through use of sequence specific hybridization techniques for example, as would be readily apparent to one of skill in the art.

Such recombinant constructs and host cells may be used to deliver wild type copies of at least one gene encoding an miRNA, and preferably the at least one genes encoding 124a miRNA, into the subject. However, any functional derivative of the miRNA may be utilised provided the desired therapeutic effect is achieved.

Any suitable vector for delivery of functional copies of at least one gene encoding an miRNA, and preferably the at least one genes encoding 124a miRNA, may be utilised according to the method of the invention where expression of the miRNA occurs in vivo. One principal requirement is that tissue specificity of delivery and expression is achieved. The two major sources of vectors which may be utilised comprise viral vectors and non-viral vectors.

Within the group of viral vectors, preferred types include adenoviruses, retroviruses, in particular Moloney murine leukaemia virus (Mo-MLV), adeno-related viruses and herpes simplex virus type I. Typically, the gene of interest, in this case encoding at least one miRNA, and preferably at least 124a miRNA, will be included in the viral genome, preferably in the “non-essential” region of the viral genome. In addition, it is important to remove virally encoded proto-oncogenes from the viral vector genome. The virus may be made replication incompetent to prevent unwanted replication once the virus has been targeted.

In terms of targeting the viral vector to the desired site, which depends upon the specific disease to be treated, a number of possibilities exist. For example, the env gene (which encodes the viral vector's envelope) may be engineered or replaced with the env gene from a different virus to alter the range of cells the viral vector will “infect”. Furthermore, alteration of the viral tropism may be achieved by using suitable antibodies raised against antigenic determinants on the cell surface of the desired target cells. The antibodies, which include all derivatives thereof, such as scFV, nanobodies, VH domains, Fab fragments etc., may be genetically incorporated into the viral vectors to provide targeted gene delivery of the at least one gene encoding an miRNA, and preferably the at least one genes encoding 124a miRNA. Most preferred is use of scFV (Hedley et al., Gene Therapy (2006) 13, 88-94). The viral vectors may have many genes removed, such as packaging genes, in order to reduce immunogenicity and/or infectivity. These functions may thus be supplied by a helper virus.

Due to their high efficiency of integration, low pathogenicity and high efficacy, adenoviruses are a preferred vector according to the methods of the invention.

Alternatives to viral vectors include direct gene delivery, use of other delivery agents and use of molecular conjugates. Tissue specific promoters may be employed as appropriate. Direct gene delivery may be achieved for example by microinjection of a suitable vector, such as a recombinant construct of the invention, directly into the tissue of interest. Alternatives include use of ballistic transformation, for example using vector coated onto suitable particles (e.g. gold particles). Additional delivery agents include liposomes and derivatives thereof. As discussed above, targeting proteins such as antibodies and derivatives thereof may be utilised in order to ensure delivery to the cells of interest. Molecular conjugates may include suitable proteins conjugated to the DNA of interest using a suitable DNA binding agent.

It is important that the nucleic acid molecules or gene copies provided in the recombinant constructs, host cells and other vectors of the invention are unmethylated in order to ensure maximal expression of the miRNA to provide a therapeutic effect. One method of preventing methylation of the miRNA encoding nucleic acid is to also incorporate a sequence encoding an inhibitor of DNA methyltransferase activity into the recombinant construct. Thus, suitable antisense or siRNA molecules may also be encoded by the recombinant construct for example, which when expressed down regulate any DNA methyltransferase activity present by RNA interference. Down regulation may occur through DNMT mRNA destruction or prevention of DNMT translation dependent upon the RISC complex involved. Alternatively expression may occur in suitable DKO cells, as described in more detail in the experimental section below, which include a double knockout of both DNMT1 and DNMT3b and which are therefore hypomethylated.

In a related aspect, the invention provides for the use of a vector or host cell carrying at least one gene encoding an miRNA, and preferably the at least one gene encoding 124a miRNA, in the manufacture of a medicament for treating a condition in which an miRNA is methylated, and preferably cancer, in a subject. This may also be expressed as a vector or host cell carrying at least one gene encoding an miRNA for use in treating a condition, such as cancer, in which an miRNA is methylated. Preferably, the subject has been selected for treatment according to the methods of the invention. Methylation of the at least one gene encoding an miRNA, and preferably the at least one genes encoding 124a miRNA and/or a decreased level of miRNA expression selects the subject for treatment. Thus, methylation may, in one embodiment, be determined at the level of gene expression.

In a preferred embodiment, the reduced level or activity of the miRNA, in particular 124a miRNA, is detected by determining the methylation status of the at least one gene encoding an miRNA, and preferably the at least one genes encoding 124a miRNA. This may be done according to any of the methods of the invention described. Thus, a particular subgroup of subjects suffering from, or predicted to have a likelihood of developing, a particular disease such as cancer is selected for treatment according to whether the at least one gene encoding an miRNA, and preferably the at least one genes encoding 124a miRNA, is methylated or not (which may be determined at the level of gene expression if desired).

As mentioned above, in a particularly preferred embodiment of the invention, the disease which is treated is cancer. Preferably, the disease or cancer is not blastoma, in particular neuroblastoma, or sarcoma. In a particularly preferred embodiment of the invention, the cancer is selected from colon cancer, breast cancer, lung cancer, leukaemia and lymphoma. A most preferred disease condition in which methylation of miRNA genes is relevant and in which patients may be selected and treated in accordance with the methods of the invention is colon cancer.

For all of the relevant aspects (such as methods of treatment) of the invention, as appropriate the DNA demethylating agent may be any agent capable of up regulating transcription of at least one miRNA, and preferably at least 124a miRNA. A preferred DNA demethylating agent comprises, consists essentially of or consists of a DNA methyltransferase inhibitor. For all of the relevant methods of the invention, the DNA methyltransferase inhibitor may be any suitable inhibitor of DNA methyltransferase which is suitable for treating cancer in the presence of methylation of the at least one gene encoding an miRNA, and preferably the at least one genes encoding 124a miRNA. As is shown in the experimental section below, methylation of the genes encoding 124a miRNA is linked to the incidence of cancer and so preventing this methylation is predicted to help to treat cancer.

The DNA methyltransferase inhibitor may, in one embodiment, be one which reduces expression of DNMT genes, such as suitable antisense molecules, or siRNA molecules which mediate RNAi for example. The design of a suitable siRNA molecule is within the capability of the skilled person and suitable molecules can be made to order by commercial entities (see for example, www.ambion.com). Preferably, the DNA methyltransferase gene is (human) DNMT1.

Alternatively, the agent may be a direct inhibitor of DNMTs. Examples include modified nucleotides such as phosphorothioate modified oligonucleotides (as set out in FIG. 6 of Villar-Garea, A. And Esteller, M. DNA demethylating agents and chromatin-remodelling drugs: which, how and why? Current Drug Metabolism, 2003, 4, 11-31, which reference is hereby incorporated in its entirety) and nucleosides and nucleotides such as cytidine analogues. Suitable examples of cytidine analogues include 5-azacytidine, 5-aza-2′-deoxycytidine, 5-fluoro-2′-deoxycytidine, pseudoisocytidine, 5,6-dihydro-5-azacytidine, 1-β-D-arabinofuranosyl-5-azacytosine (known as fazabarine) (see FIG. 4 of Villar-Garea, A. And Esteller, M. DNA demethylating agents and chromatin-remodelling drugs: which, how and why? Current Drug Metabolism, 2003, 4, 11-31, which reference is hereby incorporated in its entirety).

In another embodiment, the DNA methyltransferase inhibitor comprises Decitabine. Full details of this drug can be found at www.supergen.com for example.

Additional DNMT inhibitors include S-Adenosyl-Methionine (SAM) related compounds like ethyl group donors such as L-ethionine and non-alkylating agents such as S-adenosyl-homocysteine (SAH), sinefungin, (S)-6-methyl-6-deaminosine fungin, 6-deaminosinefungin, N4-adenosyl-N4-methyl-2,4-diaminobutanoic acid, 5′-methylthio-5′-deoxyadenosine (MTA) and 5′-amino-5′-deoxyadenosine (Villar-Garea, A. And Esteller, M. DNA demethylating agents and chromatin-remodelling drugs: which, how and why? Current Drug Metabolism, 2003, 4, 11-31).

Further agents which may alter DNA methylation and which may, therefore, be useful in the present compositions include organohalogenated compounds such as chloroform etc, procianamide, intercalating agents such as mitomycin C, 4-aminobiphenyl etc, inorganic salts of arsenic and selenium and antibiotics such as kanamycin, hygromycin and cefotaxim (Villar-Garea, A. And Esteller, M. DNA demethylating agents and chromatin-remodelling drugs: which, how and why? Current Drug Metabolism, 2003, 4, 11-31).

However, any suitable DNA methyltransferase inhibitor which is capable of increasing the expression of at least one gene encoding an miRNA, and preferably the at least one genes encoding 124a miRNA, and thus can contribute to the treatment of cancer, is included within the scope of the invention.

Particularly preferred DNMT inhibitors in the present invention comprise, consist essentially of or consist of 5-azacytidine and/or zebuline.

For all of the relevant methods of the invention, the histone deacetylase (HDAC) inhibitor may be any suitable inhibitor of HDAC activity which is suitable for treating cancer in the presence of methylation of at least one gene encoding an miRNA, and preferably the at least one genes encoding 124a miRNA.

In a preferred embodiment, the histone deacetylase (HDAC) inhibitor comprises at least one of trichostatin A (TSA), suberoyl hydroxamic acid (SBHA), 6-(3-chlorophenylureido)caproic hydroxamic acid (3-CI-UCHA), m-carboxycinnamic acid bishydroxylamide (CBHA), suberoylanilide hydroxamic acid (SAHA), azelaic bishydroxamic acid (ABHA), pyroxamide, scriptaid, aromatic sulfonamides bearing a hydroxamic acid group, oxamflatin, trapoxin, cyclic-hydroxamic-acid containing peptides, FR901228, MS-275, MGCD0103 (see www.methylgene.com), short-chain fatty acids and N-acetyldinaline (Villar-Garea, A. And Esteller, M. DNA demethylating agents and chromatin-remodelling drugs: which, how and why? Current Drug Metabolism, 2003, 4, 11-31).

The methods of treatment and medical uses aspects of the invention may incorporate any and all of the preferred aspects described in respect of the other methods of the invention as described above. Preferably, the diagnostic methods and/or the pharmacogenetic methods and/or the treatment regimen methods of the invention are carried out as a prelude to, or as an integral part of the methods of treating cancer according to the gene therapy aspects of the invention. The gene therapy methods may be synergistically combined with those of the methods of treatment according to the invention.

Thus, for example, the description of suitable methods for determining methylation levels of at least one gene encoding an miRNA, and preferably the at least one genes encoding 124a miRNA, suitable test samples, preferred subjects and specific types of cancer which may be treated all apply mutatis mutandis to these aspects of the invention and are not repeated here simply for reasons of conciseness.

Due to the effect of 124a miRNA on the oncogene CDK6's activity, the invention provides a method of treating cancer in a patient comprising reducing CDK6 levels and/or activity. Preferred cancer types are described above and may be selected from colon cancer, breast cancer, lung cancer, leukaemia and lymphoma, most preferably colon cancer. In a preferred embodiment of this method of the invention, the levels and/or activity of CDK6 are reduced by removing methylation of one or more genes encoding specified miRNA molecules which act to inhibit expression of CDK6 and/or by administering to the patient one or more miRNAs in an effective amount to the patient, wherein the administered miRNAs act to reduce the levels and/or activity of CDK6 in the patient. In a most preferred embodiment, the one or more genes encoding miRNA molecules whose methylation is removed comprises one or more genes encoding 124a miRNA. Similarly, preferably the one or more miRNA molecules which are administered comprise 124a miRNA (SEQ ID NO: 1 ACCGUMGUGGCGCACGGAAUU) or a functional derivative thereof with the ability to down-regulate CDK6 (protein) expression. Functional derivatives include all those retaining the ability to repress CDK6 translation, as discussed in greater detail above. The methods may be considered as medical use embodiments, in accordance with the other methods of the invention.

In a related aspect of the invention, there is provided a pharmaceutical composition comprising an miRNA together with a suitable carrier/excipient/diluent. Prior to the present invention, down-regulation of miRNA expression due to methylation had not been proven with a disease association. Accordingly, the inclusion of an miRNA in a pharmaceutical composition represents a preferred aspect of the present invention in order to allow recovery of miRNA expression levels in a subject in need of treatment. Preferably, the pharmaceutical composition includes 124a miRNA (SEQ ID NO: 1 ACCGUMGUGGCGCACGGAAUU) or a functional derivative thereof with the ability to down-regulate CDK6 expression, preferably protein expression (as defined above). In a further embodiment, the composition comprises a recombinant construct of the invention and/or a host cell of the invention and/or a vector of the invention.

It should be noted that, for the purposes of the present invention, the designation of a particular miRNA containing or producing composition is considered to encompass all pharmaceutically acceptable forms of the active compound which are useful in methods of treating conditions where specific genes encoding miRNAs are methylated, such as cancer. Thus, stereoisomers, enantiomers, salts, esters etc are all encompassed within the scope of the invention as appropriate.

Thus, in a pharmaceutical composition of the invention, preferred compositions include pharmaceutically acceptable carriers including, for example, non-toxic salts, sterile water or the like. A suitable buffer may also be present allowing the compositions to be lyophilized and stored in sterile conditions prior to reconstitution by the addition of sterile water for subsequent administration. The carrier may also contain other pharmaceutically acceptable excipients for modifying other conditions such as pH, osmolarity, viscosity, sterility, lipophilicity, somobility or the like. Pharmaceutical compositions which permit sustained or delayed release following administration may also be used. The composition may optionally incorporate suitable RNase inhibitors to prevent degradation of the miRNAs.

Suitable pharmaceutical compositions for use in the treatment methods or medical uses of the invention may be used together with other standard chemotherapeutic treatments which target tumour cells directly. Non limiting examples include paclitaxel, cyclaphosphomide and 5-tumor-uracil (5-FU) and pharmaceutically acceptable derivatives thereof including salts, etc.

The miRNA containing pharmaceutical composition may, for example, be encapsulated and/or combined with suitable carriers in solid dosage forms for oral administration which would be well known to those of skill in the art or alternatively with suitable carriers for administration in an aerosol spray. Examples of oral dosage forms include tablets, capsules and liquids.

Alternatively, the therapeutic agent may be administered parenterally. Specific examples include intradermal injection, subcutaneous injection (which may advantageously give slower absorption of the therapeutic agent), intramuscular injection (which can provide more rapid absorption), intravenous delivery (meaning the drug does not need to be absorbed into the blood stream from elsewhere), sublingual delivery (for example by dissolving of a tablet under the tongue or by a sublingual spray), rectal delivery, vaginal delivery, topical delivery, transdermal delivery and inhalation.

Furthermore, as would be appreciated by the skilled practitioner, the specific dosage regime may be calculated according to the body surface area of the patient or the volume of body space to be occupied, dependent on the particular route of administration to be used. The amount of the composition actually administered will, however, be determined by a medical practitioner based on the circumstances pertaining to the disorder to be treated, such as the severity of the symptoms, the age, weight and response of the individual.

Thus, in a related aspect there is also provided miRNA molecules for use as a medicament. Thus, specific miRNA molecules may be of therapeutic use to treat specific conditions where methylation of an miRNA encoding gene leads to lower levels of expression of the miRNA. In a more specific embodiment, there is provided 124a miRNA (SEQ ID NO: 1 ACCGUMGUGGCGCACGGAAUU) or a functional derivative thereof with the ability to down-regulate CDK6 (protein) expression, for use as a medicament. 124a miRNA is a most preferred miRNA for incorporation into a medicament due to the link shown herein between methylation of the different genes encoding this miRNA and various types of cancer. Similarly, the invention therefore also provides for use of an miRNA in the manufacture of a medicament for the treatment of cancer. Preferably, and as discussed in more detail above, the cancer is selected from colon cancer, breast cancer, lung cancer, leukaemia and lymphoma. The cancer is most preferably colon cancer. Preferably, the miRNA comprises 124a miRNA (SEQ ID NO: 1 ACCGUAAGUGGCGCACGGAAUU) or a functional derivative thereof with the ability to down-regulate CDK6 (protein) expression.

### Kits of the Invention

The invention also provides kits which may be used in order to carry out the methods of the invention. The kits may incorporate any of the preferred features mentioned in connection with the various methods and uses of the invention above, which discussion applies here mutatis mutandis.

Thus, a kit is provided for:
(a) diagnosing a disease in which an miRNA is expressed at a lower level; and/or
(b) predicting the likelihood of successful treatment of a disease in which an miRNA is expressed at a lower level and/or the likelihood of resistance to treatment of a disease in which an miRNA is expressed at a lower level with a DNA demethylating agent and/or a DNA methyltransferase inhibitor and/or a HDAC. inhibitor, and/or
(c) selecting a suitable treatment regimen for a disease in which an miRNA is expressed at a lower level
   comprising carrier means containing therein a set of primers for use in detecting the methylation status of at least one gene encoding the miRNA which is expressed at a lower level in the disease. As aforementioned, the most preferred miRNA is 124a miRNA.

This kit is preferably a kit for use in MSP and even more preferably a real-time or end point detection version of MSP.

Thus, the kit includes suitable primers for determining whether the at least one gene encoding an miRNA, and preferably the at least one genes encoding 124a miRNA is methylated. These primers may comprise any of the primers discussed in detail in respect of the various methods of the invention which may be employed in order to determine the methylation status of the at least one gene encoding an miRNA, and preferably the at least one genes encoding 124a miRNA. As mentioned above, functional derivatives of the specific primers may also be incorporated into the kits of the invention.

In one embodiment, the kit of the invention further comprises a reagent which modifies unmethylated cytosine in a detectable fashion whilst leaving methylated cytosine residues in tact. Such a reagent is useful for distinguishing methylated from unmethylated cytosine residues. In a preferred embodiment, the reagent comprises bisulphite, disulphite or hydrogen sulphite and preferably sodium bisulphite. This reagent is capable of converting unmethylated cytosine residues to uracil whereas methylated cytosines remain unconverted. This difference in residue may be utilised to distinguish between methylated and unmethylated nucleic acid in a downstream process, such as PCR using primers which distinguish between cytosine and uracil (cytosine pairs with guanine, whereas uracil pairs with adenine).

In a real-time and/or end point detection embodiments, the kit may further comprise probes for real-time detection of amplification products. These probes may simply be used to monitor progress of the amplification reaction in real-time and/or they may also have a role in determining the methylation status of the at least one gene encoding an miRNA, and preferably the at least one genes encoding 124a miRNA, themselves. Thus, the probes may be designed in much the same fashion as the primers to take advantage of sequence differences following treatment with a suitable reagent such as sodium bisulphite dependent upon the methylation status of the appropriate cytosine residues (found in CpG dinucleotides).

The probes may comprise any suitable probe type for real-time detection of amplification products. Non-limiting examples include hairpin primers (Amplifluor)/hairpin probes (Molecular Beacons)/hydrolytic probes (Taqman)/FRET probe pairs (Lightcycler)/primers incorporating a hairpin probe (Scorpion)/primers incorporating complementary sequences of DNAzymes that cleave a reporter substrate included in the reaction mixture (DzyNA®/fluorescent dyes (SYBR Green etc.)/oligonucleotide blockers/the specific interaction between two modified nucleotides (Plexor).

All of these technologies are well characterised in the art and the design of suitable probes is routine for one of skill in the art. Notably, however, with the AMPLIFLUOR and SCORPION embodiments, the probes are an integral part of the primers which are utilised. The probes are typically fluorescently labelled, although other label types may be utilised as appropriate.

In one embodiment, the primers in the kit comprise, consist essentially of, or consist of primers which are capable of amplifying methylated and/or unmethylated DNA following bisulphite treatment which DNA comprises, consists essentially of, or consists of the nucleotide sequence of the gene or genes encoding 124a miRNA. These genes are found at the genomic loci 8p23.1/8q12.3/20q13.33 and are known as MIRN124A1, MIRN124A2 and MIRN124A3 respectively.

In a specific embodiment, the primers in the kit comprise, consist essentially of, or consist of primers comprising, consisting essentially of, or consisting of the following nucleotide sequences for the purposes of amplifying methylated DNA (following bisulphite treatment):

| | |
|---|---|
| Genomic locus 1-8p23.1 | |
| miR-124a1 Methylated Sense | |
| AAAGAGTTTTTGGAAGACGTC | (SEQ ID NO: 2) |
| miR-124a1 Methylated Antisense | |
| AATAAAAAACGACGCGTATA | (SEQ ID NO: 3) |
| Genomic locus 2-8q12.3 | |
| miR-124a2 Methylated Sense | |
| GGGTAATTAATTTGGATTTACGTC | (SEQ ID NO: 6) |
| miR-124a2 Methylated Antisense | |
| ACCGCTATTAATTAATCTATTCCG | (SEQ ID NO: 7) |
| Genomic locus 3-20q13.33 | |
| miR-124a3 Methylated Sense | |
| GCGAGGATTTTACGTAAGTTC | (SEQ ID NO: 10) |
| miR-124a3 Methylated Antisense | |
| CCGCGTACCTTAATTATATAA | (SEQ ID NO: 11) |

In a specific embodiment, the primers in the kit comprise, consist essentially of, or consist of primers comprising, consisting essentially of, or consisting of the following nucleotide sequences for the purposes of amplifying unmethylated DNA (following bisulphite treatment):

| | |
|---|---|
| Genomic locus 1-8p23.1 | |
| miR-124a1 Unmethylated Sense | |
| AATAAAGAGTTTTTGGAAGATGTT | (SEQ ID NO: 4) |
| miR-124a1 Unmethylated Antisense | |
| AAAAAAATAAAAAACAACACATATAC | (SEQ ID NO: 5) |
| Genomic locus 2-8q12.3 | |
| miR-124a2 Unmethylated Sense | |
| GGGGTAATTAATTTGGATTTATGTT | (SEQ ID NO: 8) |
| miR-124a2 Unmethylated Antisense | |
| AAAACCACTATTAATTAATCTATTCCA | (SEQ ID NO: 9) |
| Genomic locus 3-20q13.33 | |
| miR-124a3 Unmethylated Sense | |
| GGGTGAGGATTTTATGTAAGTTT | (SEQ ID NO: 12) |
| miR-124a3 Unmethylated Antisense | |
| TTCACCACATACCTTAATTATATAAAC | (SEQ ID NO: 13) |

Functional derivatives of these sequences (as defined herein) are explicitly included within the scope of the invention.

In a further embodiment, in which bisulphite sequencing is utilised in order to determine the methylation status of the at least one gene encoding an miRNA, and preferably the at least one genes encoding 124a miRNA, the kit comprises primers for use in sequencing through the important CpG islands in the at least one gene encoding an miRNA, and preferably the at least one genes encoding 124a miRNA. Thus, primers may be designed in both the sense and antisense orientation to direct sequencing across the promoter region of the gene.

In one embodiment, the primers in the kit comprise, consist essentially of, or consist of primers which are capable of sequencing of DNA following bisulfite treatment which DNA comprises, consists essentially of, or consists of the nucleotide sequence of the gene or genes encoding 124a miRNA. These genes are found at the genomic loci 8p23.1/8q12.3/20q13.33 and are known as MIRN124A1, MIRN124A2 and MIRN124A3 respectively.

In one embodiment, bisulphite sequencing may be carried out by using sequencing primers which comprise, consist essentially of or consist of the following sequences, and which may be used in isolation or in combination to sequence both strands:

| | |
|---|---|
| Genomic locus 1-8p23.1 | |
| miR-124a1 Sense | |
| AAGGATGGGGGAGAATAAAGAGTTT | (SEQ ID NO: 14) |
| miR-124a1 Antisense | |
| CTCAACCAACCCCATTCTTAACATT | (SEQ ID NO: 15) |
| Genomic locus 2-8q12.3 | |
| miR-124a2 Sense | |
| GGTAATGGTTATGAYGGAGAATATGT | (SEQ ID NO: 16) |
| miR-124a2 Antisense | |
| CCAACTCCTATCTCTACTCATCTC | (SEQ ID NO: 17) |
| Genomic locus 3-20q13.33 | |
| miR-124a3 Sense | |
| GGAAAGGGGAGAAGTGTGGGTTTT | (SEQ ID NO: 18) |
| miR-124a3 Antisense | |
| RAAAACRCCTCTCTTAACATTCACC | (SEQ ID NO: 19) |

Again, functional derivatives of these sequences, which retain usefulness in bisulphite sequencing (of one or more genes encoding 124a miRNA) are included within the scope of the invention. Thus, the discussion above applies mutatis mutandis here.

As discussed with respect to the methods of the invention, suitable controls may be utilised in order to act as quality control for the methods. Accordingly, in one embodiment, the kit of the invention further comprises, consists essentially of or consists of one or more control nucleic acid molecules of which the methylation status is known. These (one or more) control nucleic acid molecules may include both nucleic acids which are known to be, or treated so as to be, methylated and/or nucleic acid molecules which are known to be, or treated so as to be, unmethylated. One example of a suitable internal reference gene, which is generally unmethylated, but may be treated so as to be methylated, is β-actin.

Furthermore, the kit of the invention may further comprise, consist essentially of or consist of primers for the amplification of the control nucleic acid. These primers may be the same primers as those utilised to monitor methylation in the test sample in a preferred embodiment. Thus, the control nucleic acid may comprise at least one gene encoding an miRNA, and preferably the at least one genes encoding 124a miRNA, for example taken from normal tissues (such as normal colon) in which it is known to be unmethylated. The control nucleic acid may additionally comprise at least one gene encoding an miRNA, and preferably the at least one genes encoding 124a miRNA in methylated form, for example as methylated by a methyltransferase enzyme such as SssI methyltransferase for example.

Suitable probes for use in determining the methylation status of the control nucleic acid molecules may also be incorporated into the kits of the invention. The probes may comprise any suitable probe type for real-time or end-point detection of amplification products. Non-limiting examples include hairpin primers (Amplifluor)/hairpin probes (Molecular Beacons)/hydrolytic probes (Taqman)/FRET probe pairs (Lightcycler)/primers incorporating a hairpin probe (Scorpion)/primers incorporating complementary sequences of DNAzymes that cleave a reporter substrate included in the reaction mixture (DzyNA®/fluorescent dyes (SYBR Green etc.)/oligonucleotide blockers/the specific interaction between two modified nucleotides (Plexor). All of these technologies are well characterised in the art and the design of suitable probes is routine for one of skill in the art. Notably, however, with the AMPLIFLUOR and SCORPION embodiments, the probes are an integral part of the primers which are utilised. Similarly, kits may include SYBR Green reagent to allow real time or end point detection of amplification products.

The kits of the invention may additionally include suitable buffers and other reagents for carrying out the claimed methods of the invention. Thus, the discussion provided in respect of the methods of the invention as to the requirements for determination of the methylation status of at least one gene encoding an miRNA, and preferably the at least one genes encoding 124a miRNA, apply mutatis mutandis here.

In one embodiment, the kit of the invention further comprises, consists essentially of, or consists of nucleic acid amplification buffers.

The kit may also additionally comprise, consist essentially of or consist of enzymes to catalyze nucleic acid amplification. Thus, the kit may also additionally comprise, consist essentially of or consist of a suitable polymerase for nucleic acid amplification.

Examples include those from both family A and family B type polymerises, such as Taq, Pfu, Vent etc.

In a related aspect of the invention, a kit is provided for:
(a) diagnosing a disease in which an miRNA is expressed at a lower level; and/or
(b) predicting the likelihood of successful treatment of a disease in which an miRNA is expressed at a lower level and/or the likelihood of resistance to treatment of a disease in which an miRNA is expressed at a lower level with a DNA demethylating agent and/or a DNA methyltransferase inhibitor and/or a HDAC inhibitor, and/or
(c) selecting a suitable treatment regimen for a disease in which an miRNA is expressed at a lower level
   comprising carrier means containing therein a set of primers for use in detecting the expression level of an miRNA which is expressed at a lower level in the disease. As aforementioned, the most preferred miRNA is 124a miRNA. The most preferred disease is cancer, as discussed above. Specific preferred cancers are colon cancer, breast cancer, lung cancer, leukaemia and lymphoma, preferably colon cancer.

This kit is preferably a kit for use in RT-PCR and even more preferably a real-time detection version of RT-PCR.

Thus, the kit includes suitable primers for determining whether the miRNA, and preferably 124a miRNA is expressed at a low level in the sample. These primers may comprise any of the primers discussed in detail in respect of the various methods of the invention which may be employed in order to determine the expression levels of the miRNA, and preferably at least 124a miRNA. As mentioned above, functional derivatives of the specific primers may also be incorporated into the kits of the invention.

In a real-time or end point detection embodiment, the kit may further comprise probes for real-time detection of amplification products. These probes are used to monitor progress of the amplification reaction in real-time or at end point.

The probes may comprise any suitable probe type for real-time or end point detection of amplification products. Non-limiting examples include hairpin primers (Amplifluor)/hairpin probes (Molecular Beacons)/hydrolytic probes (Taqman)/FRET probe pairs (Lightcycler)/primers incorporating a hairpin probe (Scorpion)/primers incorporating complementary sequences of DNAzymes that cleave a reporter substrate included in the reaction mixture (DzyNA®)/fluorescent dyes (SYBR Green etc.)/oligonucleotide blockers/the specific interaction between two modified nucleotides (Plexor). All of these technologies are well characterised in the art and the design of suitable probes is routine for one of skill in the art. Notably, however, with the AMPLIFLUOR and SCORPION embodiments, the probes are an integral part of the primers which are utilised. The probes are typically fluorescently labelled, although other label types may be utilised as appropriate.

In one embodiment, the primers in the kit comprise, consist essentially of, or consist of primers which are capable of amplifying reverse transcribed RNA, producing cDNA, which RNA comprises, consists essentially of, or consists of the nucleotide sequence of the pri-miRNA, pre-miRNA or mature 124a miRNA. The genes encoding the miRNAs, in various forms from primary transcript through to processed final miRNA molecules are found at the genomic loci 8p23.1/8q12.3/20q13.33 and are known as MIRN124A1, MIRN124A2 and MIRN124A3 respectively.

In a specific embodiment, the primers in the kit comprise, consist essentially of, or consist of primers comprising, consisting essentially of, or consisting of the following nucleotide sequences for the purposes of amplifying cDNA:

(SEQ ID NO: 32)

5′-GTT CAC AGC GGA CCT TGA TT-3′ (sense)
and

(SEQ ID NO: 33)

5′-ACC GCG TGC CTT AAT TGT AT-3′ (antisense) for
miR-124a.

Functional derivatives of these sequences are also envisaged. The primers may contain minor variations, such as single nucleotide substitutions, small additions and deletions, provided that their ability to act as RT-PCR primers is not compromised. Thus, variant primers must retain the ability to allow determination of expression of 124a miRNA through RT-PCR techniques. Thus, the primers may be between 10 and 50 nucleotides in length, preferably between 12 and 40 nucleotides and most preferably between 15 and 30 nucleotides.

In a preferred embodiment, the RT-PCR is carried out in real time or at end point and in a quantitative manner. Real time quantitative RT-PCR has been thoroughly described in the literature (see Gibson et al for an early example of the technique) and a variety of techniques are possible. Examples include use of hairpin primers (Amplifluor)/hairpin probes (Molecular Beacons)/hydrolytic probes (Taqman)/FRET probe pairs (Lightcycler)/primers incorporating a hairpin probe (Scorpion)/primers incorporating complementary sequences of DNAzymes that cleave a reporter substrate included in the reaction mixture (DzyNA®)/fluorescent dyes (SYBR Green etc.)/oligonucleotide blockers/the specific interaction between two modified nucleotides (Plexor). systems. All of these systems are commercially available and well characterised, and may allow multiplexing (that is, the determination of expression of multiple genes in a single sample).

In one specific embodiment, real-time RT-PCR is carried out using SYBR Green as the detection reagent. This reagent is commercially available as a PCR master mix from Applied Biosystems. In one specific embodiment, the kit for real time RT-PCR includes an antisense oligonucleotide comprising, consisting essentially of or consisting of the nucleotide sequence GCGAGCACAGAATTAATACGACTC (SEQ ID NO: 37), optionally together with a sense primer comprising, consisting essentially of or consisting of the nucleotide sequence TTA AGG CAC GCG GTG MT GCC A (miR-124a) (SEQ ID NO: 38). Again, functional derivatives which retain function in the real-time RT-PCR assay are envisaged within the kits of the invention (as defined above).

As discussed with respect to the methods of the invention, suitable controls may be utilised in order to act as quality control for the methods. Accordingly, in one embodiment, the kit of the invention further comprises, consists essentially of or consists of one or more control nucleic acid molecules of which the expression levels are known. One example of a suitable internal reference gene is β-actin.

Furthermore, the kits of the invention may further comprise, consist essentially of or consist of primers for the amplification of the control nucleic acid. These primers may be the same primers as those utilised to monitor expression in the test sample in a preferred embodiment. In one embodiment, primers for determining expression levels of β-actin are included within the kits of the invention.

The kits of the invention may additionally include suitable buffers and other reagents for carrying out the claimed methods of the invention. Thus, the discussion provided in respect of the methods of the invention as to the requirements for determination of the expression levels of at least one miRNA, and preferably at least 124a miRNA, apply mutatis mutandis here.

In one embodiment, the kit of the invention further comprises, consists essentially of, or consists of nucleic acid amplification buffers.

The kit may also additionally comprise, consist essentially of or consist of enzymes to catalyze nucleic acid amplification. Thus, the kit may also additionally comprise, consist essentially of or consist of a suitable polymerase for nucleic acid amplification. Examples include those from both family A and family B type polymerises, such as Taq, Pfu, Vent etc. The kits may also include suitable reverse transcriptase enzymes for reverse transcription of mRNA (including pri- and pre-miRNAs) and also suitable primers for priming reverse transcription.

The kits of the invention may additionally incorporate the specific components required for additional analysis by chromatin immunoprecipitation and/or for determining CDK6 expression and/or for determining Rb phosphorylation as appropriate. In these embodiments, the description above in respect of the various methods applies mutatis mutandis to the kits of the invention which may incorporate the appropriate reagents.

The kits of the invention may separately or additionally contain a pharmaceutical composition of the invention and/or a DNA methylation agent and/or a DNMT inhibitor or HDAC inhibitor for use depending upon the determination of the methylation status of at least one gene encoding an miRNA, and preferably the at least one genes encoding 124a miRNA and/or the expression levels of at least one miRNA, especially 124a miRNA. Thus, if the kits of the invention prove a positive result in terms of methylation and/or resultant down-regulation of expression, the compositions in the kit may then be employed as a means of treating the condition, which is preferably cancer as discussed and defined above.

The various components of the kit may be packaged separately in separate compartments or may, for example be stored together where appropriate.

The kit may also incorporate suitable instructions for use, which may be printed on a separate sheet or incorporated into the kit packaging for example.

The invention will now be described with respect to the following non-limiting examples.

### DESCRIPTION OF THE DRAWINGS

FIGS. 1A-1F describe epigenetic silencing of miR-124a in cancer cells.

FIG. 1A Schematic strategy used to unmask DNA methylation-associated repression of miRNAs in colon cancer cells.

FIG. 1B Bisulfite genomic analyses of miR-124a CpG island methylation status in normal colon, HCT-116 and DKO cells. Eight single clones are represented for each sample. White and black squares indicate unmethylated and methylated CpGs, respectively. Black and grey arrows indicate location of the sequencing and methylation-specific PCR primers, respectively. Transcription start site is indicated by a thick arrow.

FIG. 1C Methylation specific PCR analyses for miR-124a methylation in primary colorectal tumors. Pairs of normal colon vs colorectal tumors from three different patients are shown. The presence of a band under the U or M lanes indicates Unmethylated or Methylated sequences, respectively. Normal lymphocytes (NL) and In Vitro methylated DNA (IVD) are shown as unmethylated and methylated control sequences, respectively.

FIG. 1D Expression analyses of precursor miR-124a by conventional RT-PCR. HCT-116 cells treated with the DNA demethylating agent 5-aza-2′-deoxycytidine (AZA) and DKO cells show miR-124a upregulation.

FIG. 1E Expression analyses of mature miR-124a by quantitative RT-PCR. HCT-116 cells treated with the DNA demethylating agent 5-aza-2′-deoxycytidine (AZA) and DKO cells show miR-124a upregulation.

FIG. 1F Chromatin immunoprecipitation assay for histone modification marks and methyl-CpG binding domain proteins (MeCP2 and MBD2) in the miR-124a CpG island. The presence of miR-124a CpG island methylation is associated with the lack of histone modifications linked to transcriptional activity (acetylation of histones H3 and H4, and trimethylation of lysine 4 of histone H3) and with the occupancy by MBDs, whilst the opposite scenario is observed when DNA demethylation events are present by genetic disruption of the DNA methylatransferases (DKO cells) or pharmacological treatment with a DNA demethylating agent (AZA).

FIGS. 2A-2G describe epigenetic silencing of miR-124a leads to CDK6 activation.

FIG. 2A—Western blot for CDK6 in wild type HCT-116, HCT-116 cells treated with a DNA demethylating agent (AZA) and DKO cells. These two cell lines show a reduction in CDK6 levels.

FIG. 2B—RT-PCR analyses of CDK6 mRNA do not demonstrate any change in CDK6 levels between wild-type HCT-116 cells, AZA treated cells and DKO cells.

FIG. 2C—miR-124a interact and interfere with the 3′ UTR of CDK6 mRNA. Base pairing comparison between mature miR-124a and the wild-type (WT) or mutant (MUT) CDK6 putative target site.

FIG. 2D—miR-124a interact and interfere with the 3′ UTR of CDK6 mRNA. 8 luciferase assays of DKO (miR-124a expressor) or HCT-116 (miR-124a non-expressor) cells transfected with firefly luciferase constructs containing CDK6-WT or CDK-MUT. In the case of HCT-116 cells, the results of miR-124a cotransfection are also shown. Normalized luciferase activities are represented.

FIG. 2E—Western blots for CDK6 in original HCT-116 cells and HCT-116 cells transfected with miR-124a precursor, demonstrating CDK6 down-regulation. RT-PCR analyses of CDK6 mRNA do not demonstrate any change.

FIG. 2F—Western blot for phosphorylated Rb demonstrates that HCT-116 cells transfected with the miR-124a precursor, HCT-116 cells treated with the DNA demethylating agent, and DKO cells are hypophosphorylated at the two Rb sites mediated by CDK6.

FIG. 2G—Immunohistochemistry of CDK6 and phosphorylated Rb in human primary lung tumors. Left column, miR-124a unmethylated tumor showing loss of CDK6 expression and hypophosphorylated Rb; right column, miR-124a methylated tumor showing strong CDK6 staining and phosphorylated Rb.

FIGS. 3A-3D describe epigenetic silencing of miR-517c and miR-373.

FIG. 3A Bisulfite genomic analyses of miR-517c CpG island methylation status in normal colon, HCT116 and DKO cells. Eight single clones are represented for each sample. White and black squares indicate unmethylated and methylated CpGs, respectively. miR-517c CpG islands are methylated in normal colon.

FIG. 3B Bisulfite genomic analyses of miR-373 CpG island methylation status in normal colon, HCT116 and DKO cells. Eight single clones are represented for each sample. White and black squares indicate unmethylated and methylated CpGs, respectively. miR-373 CpG islands are methylated in normal colon.

FIG. 3C Expression analyses of precursor miR-517c by conventional RT-PCR. HCT-116 cells do not express the described miRNA and DKO cells show upregulation of miRNA.

FIG. 3D Expression analyses of mature miR-517c by quantitative RT-PCR. HCT-116 cells do not express the described miRNA and DKO cells show upregulation of miRNA.

FIG. 4A—Bisulfite genomic analyses of miR-124a2. CpG island methylation status in normal colon, HCT116 and DKO cells. Eight single clones are represented for each sample. White and black squares indicate unmethylated and methylated CpGs, respectively. miR-124a2 CpG islands are unmethylated in normal colon and DKO cells, but hypermethylated in HCT-116.

FIG. 4B—Bisulfite genomic analyses of miR-124a3. CpG island methylation status in normal colon, HCT116 and DKO cells. Eight single clones are represented for each sample. White and black squares indicate unmethylated and methylated CpGs, respectively. miR-124a3 CpG islands are unmethylated in normal colon and DKO cells, but hypermethylated in HCT-116.

FIGS. 5A-5D describe the profile of miR-124a methylation in human cancer.

FIG. 5A—Analyses of miR-124a methylation in human cancer cell lines. Red squares, methylated CpG island; green square, unmethylated CpG island.

FIG. 5B—Methylation-specific PCR analyses for miR-124a methylation in human cancer cell lines. The presence of a band under the U or M lanes indicates Unmethylated or Methylated sequences, respectively. Normal lymphocytes (NL) and In Vitro methylated DNA (IVD) are shown as negative and positive controls for unmethylated and methylated sequences, respectively.

FIG. 5C—Expression analyses of miR-124a by RT-PCR in a methylated breast cancer cell line (MCF-7) and two unmethylated neuroblastoma cell lines (LAN-1 and LAI-55N). MCF-7 cells treated with the DNA demethylating agent 5-aza-2′-deoxycytidine (AZA) show miR-124a upregulation.

FIG. 5D—Profile of miR-124a methylation in human primary malignancies.

### EXPERIMENTAL SECTION

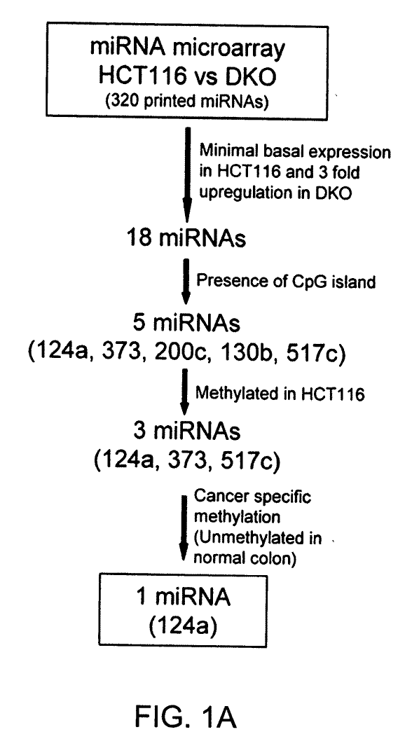

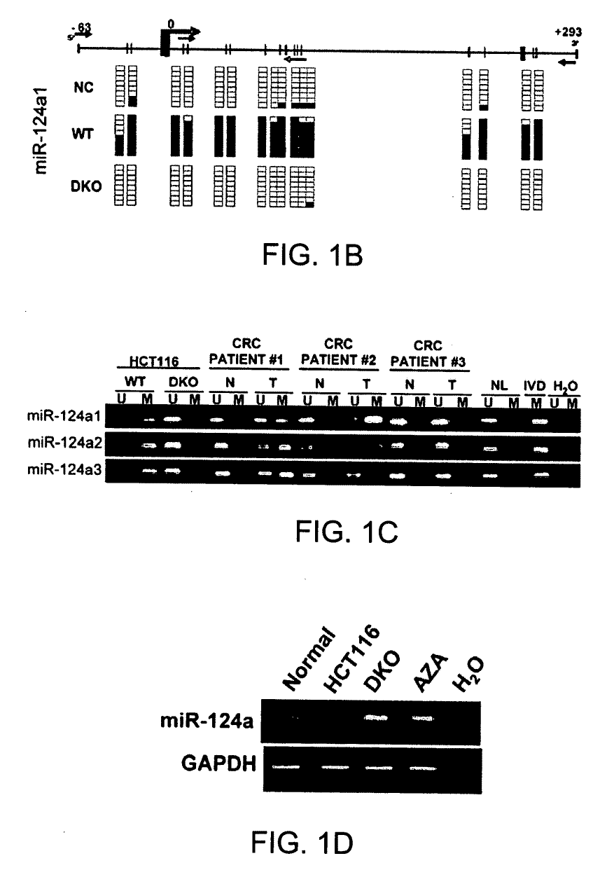

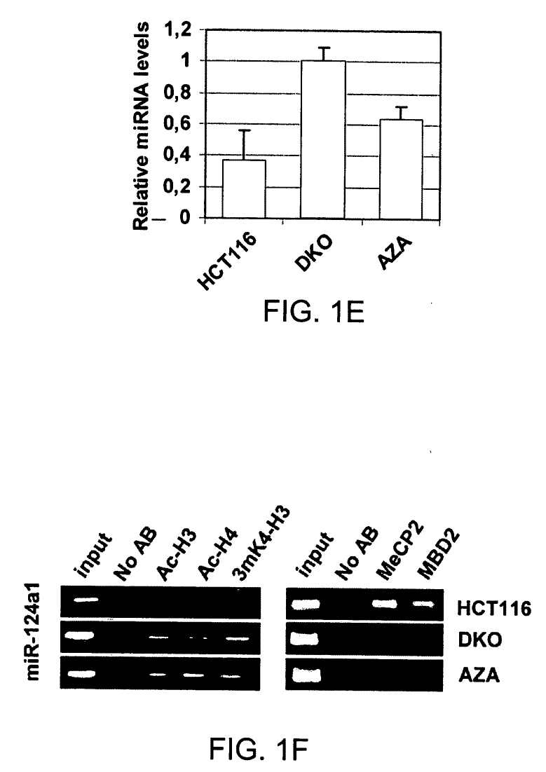

To explore the putative presence of DNA methylation associated-silencing of miRNAs in cancer cells, we used a genetic approach. We compared the miRNA expression profile of the wild-type colon cancer cell line HCT-116 with the same cell line after genetic disruption by homologous recombination of DNMT1 and DNMT3b (Double Knock-Out, DKO)(11), using a miRNA microarray expression profiling method (12). DKO cells show a drastic reduction of DNMT activity, 5-methylcytosine DNA content and, most important, a release of gene silencing associated with CpG island hypomethylation (11,13). Our genetic screening revealed that eighteen of the three hundred and twenty human miRNAs printed in the microarray showed minimal basal expression in wild-type HCT-116 cells and were upregulated more than 3-fold in DKO cells (FIG. 1a). Of these significantly upregulated miRNAs, five of them were embedded in a canonical CpG island (FIG. 1a).

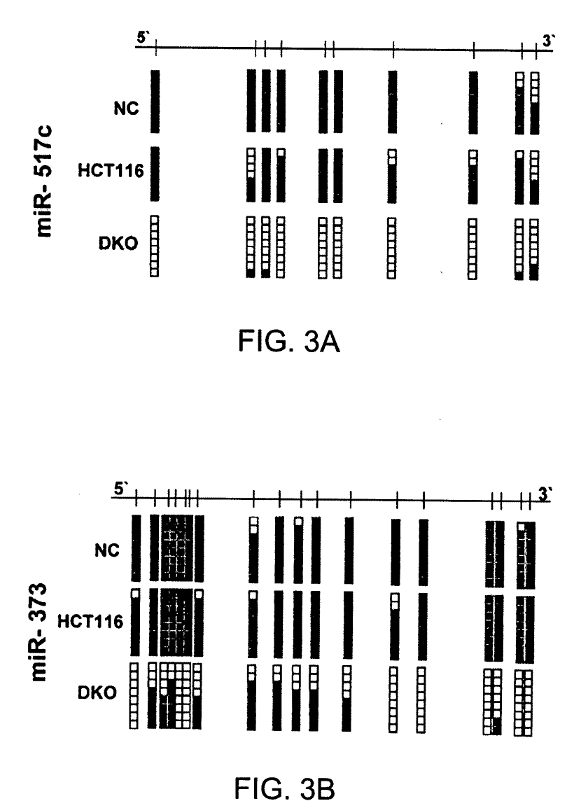

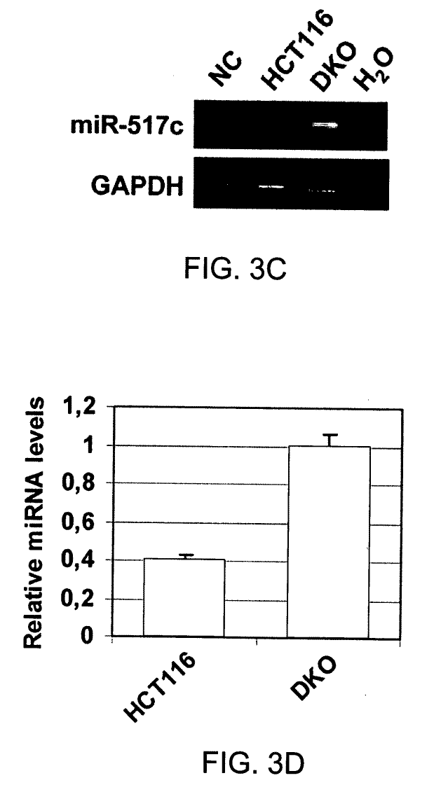

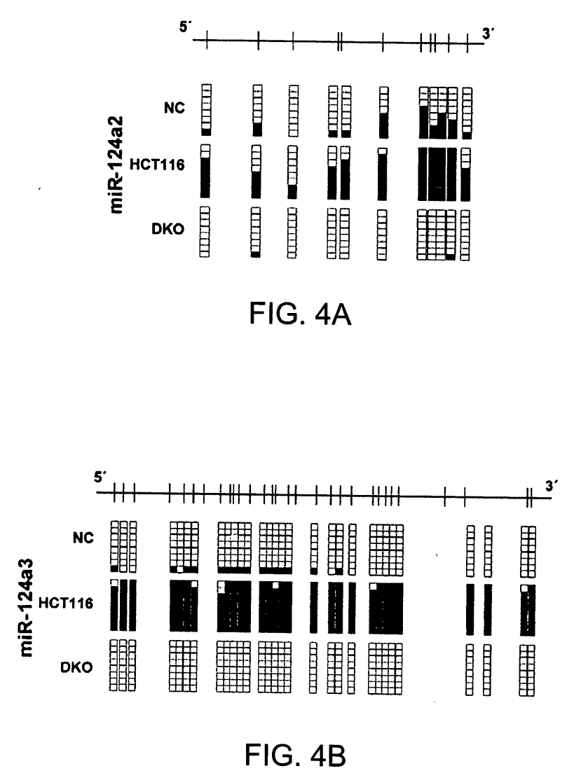

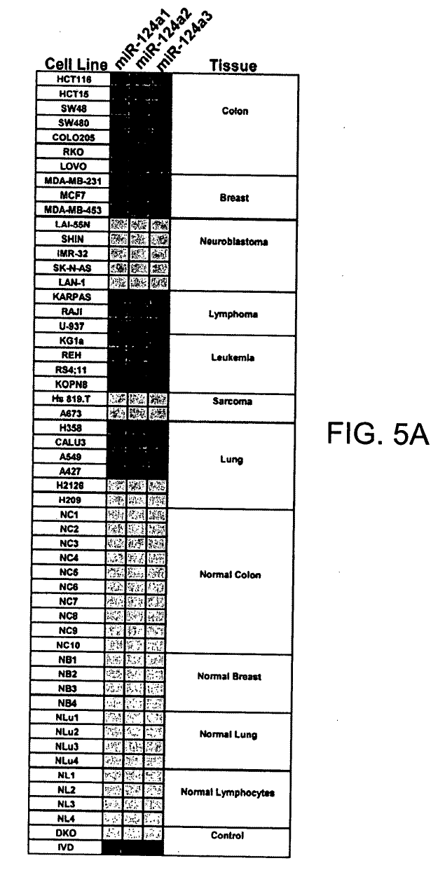

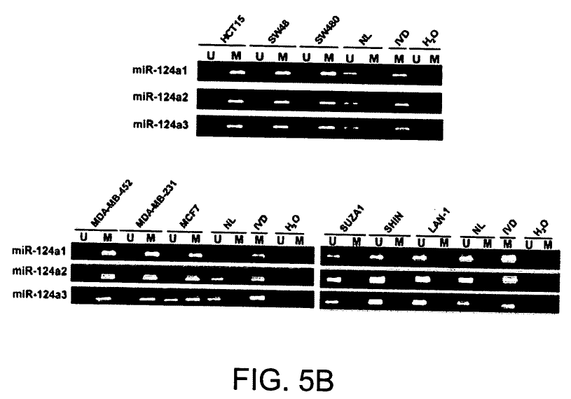

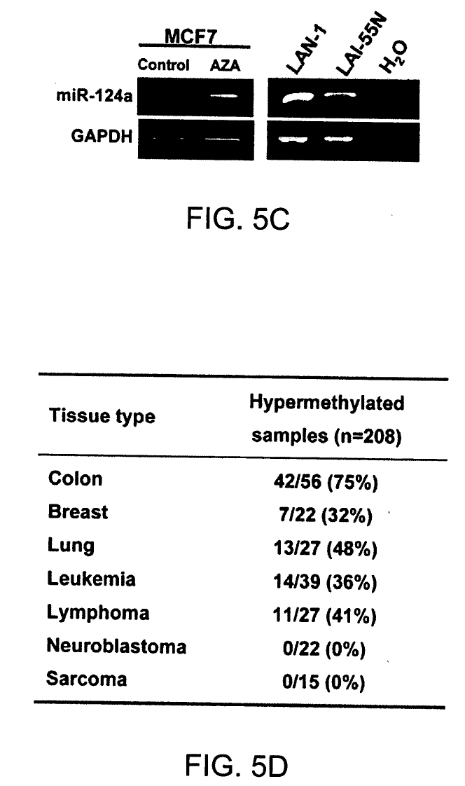

Bisulfite genomic sequencing analyses of multiple clones of the original HCT-116 cells demonstrated dense CpG island hypermethylation for miRNA-124a, miRNA-517c and miR-373 (FIGS. 1B, 3A, 3B, 4A and 4B). Because we wanted to focus in the cancer-specific DNA methylation changes, we analyzed by bisulfite genomic sequencing the DNA methylation status of these miRNA in normal colon tissues (n=10), to exclude tissue-specific methylation patterns. miRNA-517c and miRNA-373 were found to be densely methylated in normal colon tissues (FIGS. 3A and 3B), whilst miR-124a embedded CpG island was always unmethylated (FIGS. 1B, 4A and 4B). In the case of miR124, this miRNA is represented in three genomic loci miR-124a-1 (8p23.1), miR-124a-2 (8q12.3) and miR-124a-3 (20q13.33) and in all cases the corresponding CpG island was methylated in HCT-116 and unmethylated in normal colon (FIGS. 1B, 1C and FIGS. 4A and 4B). The presence of miR-124a hypermethylation was not a feature of this particular cell line, but analyzing a large set of primary human colorectal tumors was observed in 75% (42 of 56) of patients (FIG. 5D). The DNA methylation analyses of a comprehensive collection of human cancer cell lines (n=29) and primary samples (n=115) from breast and lung carcinomas, leukemias and lymphomas also demonstrated a frequent presence of miR-124a hypermethylation (FIG. 5D). However, miR-124 methylation was absent in neuroblastomas (n=22) and sarcomas (n=15) (FIG. 5). Thus, miR-124a became our prime candidate for a hypermethylation-cancer specific event.

To effectively demonstrate the epigenetic silencing of miR-124a in cancer cells, we first mapped by RACE (Supplementary Methods) the transcriptional start site of miR-124a and it was found indeed in the analyzed CpG island (FIG. 1B). Most important, RT-PCR analyses demonstrated that precursor and mature miR-124a expression was absent in HCT-116 cells with CpG island hypermethylation and restored in HCT-116 cells treated with the DNA demethylating agent and in DKO cells (FIGS. 1D and E). The association between miR-124 methylation and loss of expression was also found in cancer cell lines from other tumor types (FIGS. 5A and D). Because DNA methylation-silencing is closely linked to histone modifications to determine active vs inactive gene expression, we analyzed by chromatin immunoprecipitation the histone modification pattern and the binding of the transcriptional repressors methyl-CpG binding domain proteins (MBDs) to the miR-124a CpG island. The presence of miR-124a CpG island hypermethylation was accompanied by the absence of histone modification marks associated with gene activation such as histone H3 and H4 acetylation and trimethylation of lysine 4 of histone H3, and occupancy by MBDs, such as MeCP2 and MBD2 (FIG. 1F). The induction of miR-124a CpG island DNA hypomethylation events by the demethylating agent or in DKO cells was associated with the emergence of the histone marks of active transcription, and a release of binding by MBDs (FIG. 1F).

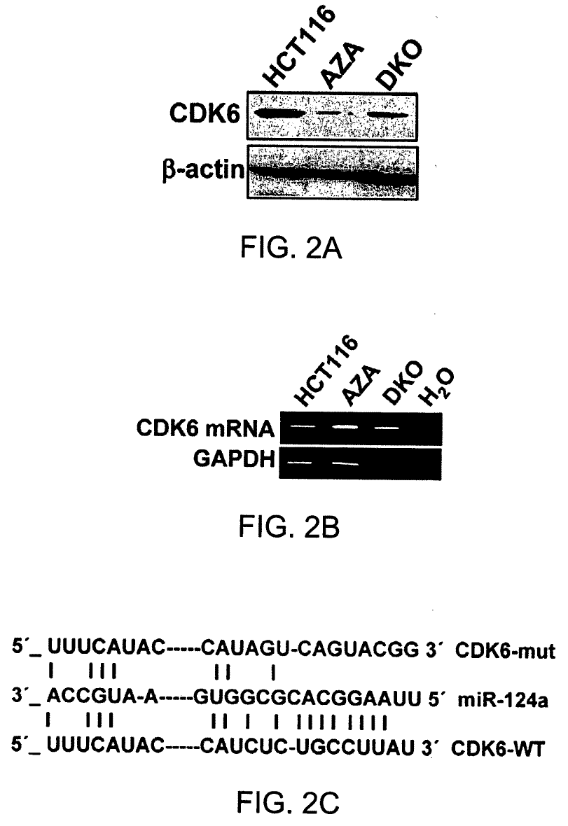

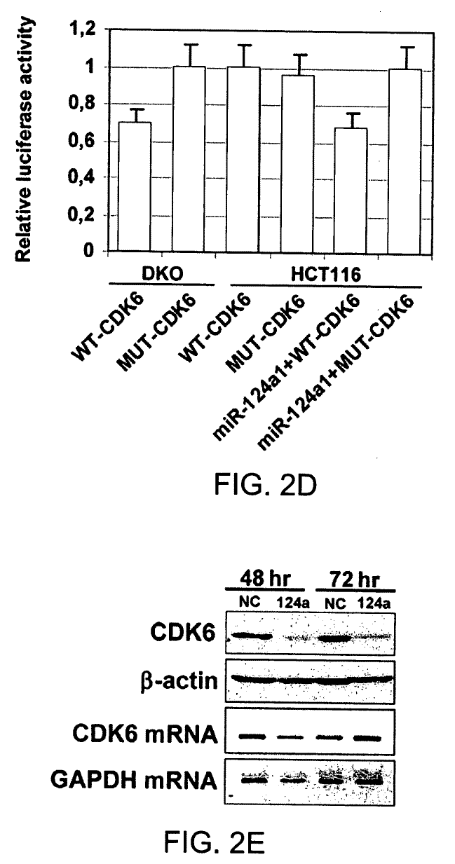

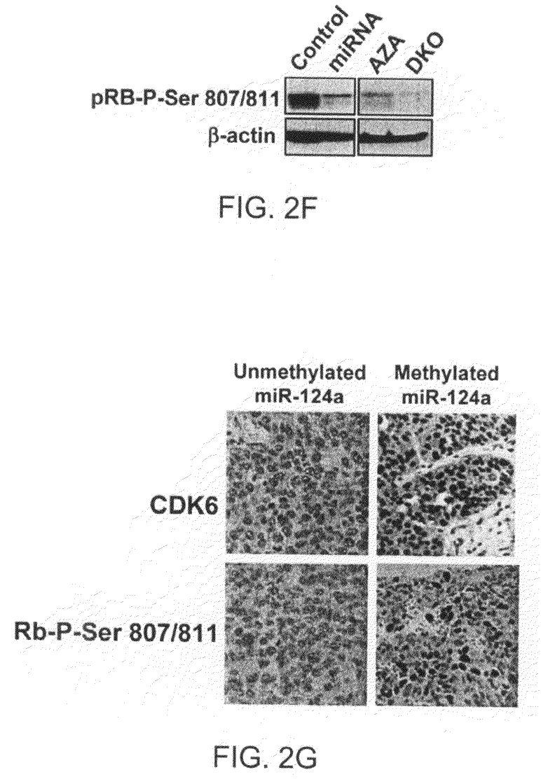

Finally, to determine whether the epigenetic silencing of miR-124a had functional cancer relevance to escape the putative tumour suppressor function of miR-124a, we examined its impact in the regulation of presumed target genes with oncogenic capacity. Using computational prediction for miR-124a target genes (Supplementary Methods), we observed that cyclin D kinase 6 (CDK6) was one of the best potential targets for miR-124a. CDK6 is involved in cell cycle progression and differentiation (14) and it constitutes an attractive target for the development of anti-cancer compounds (15). The first validation that miR-124a can target CDK6 arise from the expression studies of all the previously described colon cancer cells. Using CDK6 western-blot analyses we observed that whilst the original HCT-116 cells with miR-124a methylation-associated silencing strongly expressed CDK6, the cells treated with the demethylating agent or the DKO cells showed CDK6 downregulation (FIG. 2A). On the other hand, there was no difference in the CDK6 mRNA expression levels in any of the described cells (FIG. 2B), suggesting translational inhibition of CDK6 by miR-124a, rather than mRNA degradation or transcriptional repression. Most important, a functional link was established by performing a luciferase reporter assay with a vector containing the CDK6 wild-type (WT) putative 3′ UTR target site and a mutant form (MUT), in different contexts of miR-124a expression. Luciferase activity of miR-124a expressing-DKO cells transfected with CDK6-WT was significantly lower than DKO cells transfected with CDK6-MUT (p=0.031). In contrast, the luciferase activities of miR-124a-non expressing-HCT-116 cells transfected with CDK6-WT and CDK6-MUT showed no measurable differences (FIG. 2D). However, when we co-transfected miR-124a, luciferase activity of HCT-116 CDK6-WT transfected cells was significantly lower than CDK6-MUT (p=0.002) (FIG. 2D). To further confirm the targeting of CDK6 by miR-124a, HCT-116 cells were transfected with miR-124a precursor molecules, which are designed to mimic endogenous miR-124a and directly enter the miRNA processing pathway. Overexpression of miR-124a induced a reduction of CDK6 protein level (FIG. 2E). Most important, the miR-124a transfection diminished the phosphorylation of Rb in the residues 807 and 811, the targets of CDK614. The induction of endogenous miR-124a in HCT-116 cells by the demethylating drug or in DKO cells also reduced Rb phosphorylation (FIG. 2F). Remarkedly, the described targeting of CDK6 by miR-124a was also observed in human primary tumors. An immunostaining analyses of CDK6 expression in lung cancer patients (n=27), demonstrated that miR-124a hypermethylation was associated with strong expression of CDK6 and Rb-phosphorylation (p=0.045) (FIG. 2G). All these data indicate that CDK6 is targeted by miR-124a and that epigenetic silencing of miR-124a in cancer cells leads to CDK6 upregulation.

In summary, we have demonstrated that one mechanism accounting for the observed downregulation of miRNAs in human cancer is CpG island hypermethylation, in a similar manner that for classical tumor suppressor genes. We provide an illustrative example in colon cancer with the epigenetic silencing of miR-124a and its functional consequences for CDK6 activity. Most important, miRNA function can be restored by erasing DNA methylation, a finding that may have significant consequences for cancer patients undergoing treatment with DNA demethylating drugs in the clinical arena.

### REFERENCES

1. He, L., & Hannon, G. J. Nat. Rev. Genet. 5, 522-531 (2004).
2. Miska, E. A. Curr. Opin. Genet. Dev. 5, 563-568 (2005).
3. Lu, J. et al. Nature 435, 834-838 (2005).
4. Johnson, S. M. et al. Cell 120, 635-647 (2005).
5. Cimmino, A. et al. Proc. Natl. Acad. Sci. USA 102, 13944-13949 (2005).
6. Saito, Y. et al. Cancer Cell 9, 435-443 (2006).
7. Thomson, J. M. et al. Genes Dev. 20, 2202-2207 (2006).
8. Costello, J. F. & Plass, C. J. Med. Genet. 38, 285-303 (2001).
9. Esteller, M. Oncogene 21, 5427-5440 (2002).
10. Herman, J. G. & Baylin, S. B. N. Engl. J. Med. 349, 2042-2054 (2003).
11. Rhee, I. et al. Nature 416, 552-556 (2002).
12. Miska, E. A. et al. Genome Biol. 5, R68 (2004).
13. Paz, M. F. et al. Hum. Mol. Genet. 12, 2209-2219 (2003).
14. Grossel, M. J. & Hinds, P. W. J. Cell Biochem. 97, 485-493 (2006).
15. Hirai, H., Kawanishi, N. & Iwasawa, Y. Curr. Top. Med. Chem. 5, 167-179 (2005). 7

### Supplementary Methods

### Human Cancer Cell Lines and Primary Tumor Samples.

HCT-116 colon cancer cells were cultured in McCoy's 5A modified medium supplemented with 10% fetal bovine serum and 1% penicillin/streptomycin. HCT-116 double DNMT1−/− DNMT3b−/− (DKO) cells were grown supplemented with 0.05 mg/ml hygromycin. HCT-116 cells were treated with 5-aza-2-deoxycytidine (1 UM) for 72 h, with drug and medium replaced 24 h after the beginning of the treatment. HCT-116 and DKO cells were a generous gift from Dr Bert Vogelstein. All the other human cancer cell lines representing colon (n=7), lung (n=5), breast (n=3), leukemia (n=4), lymphoma (n=3), sarcoma (n=2) and neuroblastoma (n=6) tumor types were obtained from the ATCC and were cultured in the corresponding mediums. Primary tissue samples of colorectal (n=56), lung (n=27) and breast (n=22) cancer, leukemias (n=39), lymphomas (n=27), neuroblastomas (n=22) and sarcomas (n=15), in addition to normal colon (n=10), breast (n=4), lung (n=4) and lymphocytes (n=4), were all from specimens obtained at the time of clinically indicated procedures. DNA and RNA was extracted using standard protocols.

RNA Isolation and miRNA Expression Analysis.

Total RNA was isolated from HCT-116 and DKO cells by Trizol (Invitrogen) extraction according to the manufacturer's instructions. miRNA microarray profiling was performed as described by Miska et al (1). In brief, 5 μg of total RNA was used for each hybridization. miRNA expression levels were normalized by three different artificial miRNA spikes. Microarray probes were oligonucleotides with sequences complementary to microRNAs. Each probe was modified with a free amino group linked to its 5′ terminus through a 6-carbon spacer (IDT) and was printed onto amine-binding slides (CodeLink, Amersham Biosciences). Control probes contained two internal mismatches resulting in either C-to-G or T-to-A changes. Printing and hybridization were done using the protocols from the slide manufacturer with the following modifications: the oligonucleotide concentration for printing was 20 μM in 150 mM sodium phosphate pH 8.5, and hybridization was at 50° C. for 6 h. Printing was done using a MicroGrid TAS II arrayer (BioRobotics) at 50% humidity. DNA methylation analyses. The CpG Island Searcher Program (2) was used to determine which miRNAs were embedded in a CpG island. The DNA methylation status was established by PCR analysis of bisulfite-modified genomic DNA, which induces chemical conversion of unmethylated, but not methylated, cytosine to uracil, using two procedures. First, methylation status was analyzed by bisulfite genomic sequencing of both strands of the corresponding CpG islands. The second analysis used methylation specific PCR using primers specific for either the methylated or modified unmethylated DNA. DNA from normal lymphocytes treated in vitro with Sssl methyltransferase was used as a positive control for methylated alleles. DNA from normal lymphocytes was used as a positive control for unmethylated alleles.

Primers for bisulfite genomic sequencing:

| | |
|---|---|
| miR-124a1 Sense | |
| AAGGATGGGGGAGAATAAAGAGTTT | (SEQ ID NO: 14) |
| miR-124a1 Antisense | |
| CTCAACCAACCCCATTCTTAACATT | (SEQ ID NO: 15) |
| miR-124a2 Sense | |
| GGTAATGGTTATGAYGGAGAATATGT | (SEQ ID NO: 16) |
| miR-124a2 Antisense | |
| CCAACTCCTATCTCTACTCATCTC | (SEQ ID NO: 17) |
| miR-124a3 Sense | |
| GGAAAGGGGAGAAGTGTGGGTTTT | (SEQ ID NO: 18) |
| miR-124a3 Antisense | |
| RAAAACRCCTCTCTTAACATTCACC | (SEQ ID NO: 19) |
| miR-517c Sense | |
| GGAAGTTGAGTTTTTAGTGAGTTGAG | (SEQ ID NO: 20) |
| miR-517c Antisense | |
| AATTCTCCTACTACCRCCTCC | (SEQ ID NO: 21) |
| miR-373 Sense | |
| TTGGGGAAGGGAAGGGGGTTTT | (SEQ ID NO: 22) |
| miR-373 Antisense | |
| CCTACCTCAACCTCCCAAATAAC | (SEQ ID NO: 23) |

Primers for methylation-specific PCR:

| | |
|---|---|
| miR-124a1 Methylated Sense | |
| AAAGAGTTTTTGGAAGACGTC | (SEQ ID NO: 2) |
| miR-124a1 Methylated Antisense | |
| AATAAAAAACGACGCGTATA | (SEQ ID NO: 3) |
| miR-124a1 Unmethylated Sense | |
| AATAAAGAGTTTTTGGAAGATGTT | (SEQ ID NO: 4) |
| miR-124a1 Unmethylated Antisense | |
| AAAAAAATAAAAAACAACACATATAC | (SEQ ID NO: 5) |
| miR-124a2 Methylated Sense | |
| GGGTAATTAATTTGGATTTACGTC | (SEQ ID NO: 6) |
| miR-124a2 Methylated Antisense | |
| ACCGCTATTAATTAATCTATTCCG | (SEQ ID NO: 7) |
| miR-124a2 Unmethylated Sense | |
| GGGGTAATTAATTTGGATTTATGTT | (SEQ ID NO: 8) |
| miR-124a2 Unmethylated Antisense | |
| AAAACCACTATTAATTAATCTATTCCA | (SEQ ID NO: 9) |
| miR-124a3 Methylated Sense | |
| GCGAGGATTTTACGTAAGTTC | (SEQ ID NO: 10) |
| miR-124a3 Methylated Antisense | |
| CCGCGTACCTTAATTATATAA | (SEQ ID NO: 11) |
| miR-124a3 Unmethylated Sense | |
| GGGTGAGGATTTTATGTAAGTTT | (SEQ ID NO: 12) |
| miR-124a3 Unmethylated Antisense | |
| TTCACCACATACCTTAATTATATAAAC | (SEQ ID NO: 13) |

### RACE.

5′ Rapid Amplification of cDNA Ends (RACE) system was performed using Invitrogen kit and according to the manufacturer's instruction. Briefly, 5 μg of brain total RNA was reverse transcribed into cDNA using SuperScript™ II RT reverse transcriptase and specific reverse primers (GSP1):

| | |
|---|---|
| 124a1 GSP1: | |
| 5′-CCT TAA TTG TAT GGA C-3′ | (SEQ ID NO: 24) |
| 124a2 GSP1: | |
| 5′-ATTA AAT CAA GGT CCG-3′ | (SEQ ID NO: 25) |
| 124a3 GSP1: | |
| 5′-ATT AAA TCA AGG TCC G-3′. | (SEQ ID NO: 26) |

After that, cDNAs were amplified by PCR using Elongase® Amplification System (Invitrogen) and others specific primers (GSP2 or GSP3 for nested amplification in the cases of miR-124a1 and miR-124a2). The PCR primers are as follows:

(SEQ ID NO: 27)

124a1 GSP2: 5′-CCG CTG TGA ACA CGG AGA GA-3′

(SEQ ID NO: 28)

124a2 GSP2: 5′-GAA CAC GGA GAG CAG AGC CTC T-3′

(SEQ ID NO: 29)

124a3 GSP2: 5′-GGT CCG CTG TGA ACA CGC AGA G-3′

(SEQ ID NO: 30)

124a1 GSP3 5′-GAG GGG TGG GTA GAA GAT GG-3′
and

(SEQ ID NO: 31)

124a2 GSP3: 5′-GTC CGC TGT GAA CAC GGA GAG C-3′.

RT-PCR and Q-RT-PCR for miRNAs.

We used conventional and real-time RT-PCR to measure miRNA expression. The RT-PCR primers used for miRNA precursors were:

(SEQ ID NO: 32)

5′-GTT CAC AGC GGA CCT TGA TT-3′ (sense)
and

(SEQ ID NO: 33)

5′-ACC GCG TGC CTT AAT TGT AT-3′ (antisense) for
miR-124a;
and

(SEQ ID NO: 34)

GAAGATCTCAGGCAGTGAC (sense)
and

(SEQ ID NO: 35)

AAA CAG TAA CAC TCT AAA AGG ATG C (antisense) for
miR-517c.

GAPDH was used as internal control. PCR products were analyzed in a 3% agarose gel. Realtime PCR analysis of miRNAs was performed as previously described (Biotechniques 39:519-525). In brief, total RNA was isolated from cell lines or tissues using Trizol reagent and the small RNA fraction was further isolated using the miRVANA kit (Ambion). 500 ng of the small RNA fraction was polyadenylated by poly(A) polymerase (PAP, Ambion) at 37° C. for 1 hr in a 6 uL reaction. After phenol-chloroform extraction and ethanol precipitation the polyadenylated small RNAs were reversed transcribed according to the manufacturer's protocols using 200U SuperScript-II Reverse Transcriptase (RT, Invitrogen) and 0.5 ug poly(T) adapter (GCG AGC ACA GM TTA ATA CGA CTC ACT ATA GGT TTT TTT TTT TTV N) (SEQ ID NO:36). The small cDNAs were then treated with RNAse H at 37° C. for 1 hr and finally diluted 240 fold. Negative controls were included in both PAP and RT reactions. Moreover, two additional RT reactions were performed using half and double the input of polyadenylated small RNAs to ensure the linearity of the reverse transcription step. Finally, the melting temperature for each miRNA was experimentally determined from the thermal dissociation curves using ABI Prism 7900HT (Applied Biosystems, Foster City, Calif., USA). The real-time PCR reactions were then carried out at the optimal temperature and typically contained 5 pmol of the gene-specific primer (identical to the complete miRNA sequence listed in the miRBASE database), 5 pmol of the common antisense oligonucleotide (GCGAGCACAGAATTAATACGACTC) (SEQ ID NO: 37), 10 μL SYBR Green PCR Master Mix (Applied Biosystems) and 6 uL of the diluted cDNAs in a total volume of 20 μL. The specific sense primers used were

(SEQ ID NO: 38)

TTA AGG CAC GCG GTG AAT GCC A (miR-124a)
and

(SEQ ID NO: 39)

ATC GTG CAT CCT TTT AGA GTG T (miR-517c).

Each set of PCR reactions included a dilution series of a reference cDNA to be used for quantification. The following quality control was applied before data sets were accepted: (1) both PAP and RT negative controls gave negligible values or displayed dissociation curves that were significantly different from those in experimental samples, (2) the realtime PCR values of samples containing RT reactions programmed with different RNA inputs showed a dose response, (3) the linear fit of the real-time PCR values of the dilution series was >90%.

### Chromatin Immunoprecipitation (ChIP) Assay.

Standard ChIP assays were performed as previously described (3). In brief, cells were treated with 1% formaldehyde for 15 min. Then, chromatin was sheared with a Bioruptor™ (Diagenode) to an average length of 0.4-0.8 kb for this analysis. The following antibodies were used: anti-MeCP2 (ab3752), anti-MBD2 (ab3754), and anti-trimethyl-K4 histone H3 (ab8580) (ab1220) (Abcam) and anti-acetyl H3 (06-599) and anti-acetyl H4 (06-598) (Upstate biotechnologies). PCR amplification was performed in 20 μl with specific primers for each of the analyzed promoters.

### Primers Used:

for miR-124a1 5′-CM AGA GCC TTT GGA AGA CG-3′ (sense) (SEQ ID NO: 40) and 5′-GGA AGA GGG GTG GGT AGA AG-3′ (antisense) (SEQ ID NO: 41),
for miR-124a2 5′-GCG TGG TCC TTA AAA ACC TG-3′ (sense) (SEQ ID NO: 42) and 5′-CCA TGC CAT TTA CAG CAC AC-3′ (antisense) (SEQ ID NO: 43); and
for miR-124a3 5′-GGA GM GTG TGG GCT CCT C-3′ (sense) (SEQ ID NO: 44) and 5′-AAT CM GGT CCG CTG TGA AC-3′ (antisense) (SEQ ID NO:45). For each promoter, the sensitivity of PCR amplification was evaluated on serial dilutions of total DNA collected after sonication (input fraction). PCR amplifications were carried out with a variable number of cycles at 94° C. for 30 s, 60° C. for 30 s and 72° C. for 30 s. The amplified DNA was separated on 2% agarose gel and visualized with ethidium bromide.

### Databases and GenBank Accession Number.

The miRNA sequences were analyzed using miRBase (http://microrna.sanger.ac.uk/), University of California at Santa Cruz Human Genome Browser (http://genome.cse.ucsc.edu/) and Methyl Primer Express (Applied Biosystems). Detailed information of base pairing comparison between miR-124a and its target site in the 3′UTR of CDK6 mRNA is available at Human microRNA Targets (http://www.microrna.org/) and miRBase Targets (http://microrna.sanger.ac.uk/). The GenBank accession number of CDK6 mRNA is NM_{—}001259.

Analyses of CDK6 and phosphorylated Rb expression by Western blot, immunohistochemistry and RT-PCR. We collected cells by centrifugation and washed cell pellets twice with phosphate-buffered saline buffer. Total extracts were fractionated on a SDS-PAGE gel and transferred to a polyvinylidene difluoride membrane with 45-μm pore size (Immobilon PSQ, Millipore). The membranes were blocked in 5% milk PBS-T (phosphate-buffered saline with 0.1% Tween-20) and immunoprobed with antibodies against CDK6 (1:1000; Cell Signaling) and P-Rb-S807/811 (1:1000; Cell Signaling). An antibody to β-actin (1:5000; Sigma) was used as a loading control. Immunoreactive bands were visualized using a chemiluminescent substrate (Amersham) after incubation of the membrane with secondary antibodies conjugated to horseradish peroxidase.

Immunohistochemical staining of CDK6 and PRb-S807/811 was performed using the described above antibodies at a 1:1500 dilution. After antigen retrieval with citrate buffer, immunodetection was performed by the DAKO EnVision Visualization Method (DAKO, Glostrup, Denmark), with diaminobenzidine chromogen as the substrate. For the RT-PCR of CDK6, total RNA (5 μg) was used for reverse transcription. After incubation with DNase I (Invitrogen) to eliminate DNA contamination, Superscript III (Invitrogen) and random hexamers (Promega, Madison, Wis.) were added for first strand cDNA synthesis. Then PCR was performed with primers specific for CDK6 mRNA (forward, 5′-GCC TAT GGG MG GTG TTC M-3′ (SEQ ID NO: 46); reverse, 5′-CAC TCC AGG CTC TGG MC TT-3′) (SEQ ID NO: 47).

Transfection with miR-124a precursor molecules and luciferase assays. miR-124a precursor molecules and negative control miRNA were purchased from Ambion. Experiments involving transient transfections of miRNAs were carried out with oligofectamine (Invitrogen) using 100 nM RNA duplexes. The cells were collected 48 h after transfection and the expression of CDK6 and P-Rb-S807/811 were analyzed by Western Blot and RT-PCR, as described above. For Luciferase experiments the luciferase reporter assay system (Ambion) was used. Luciferase constructs were made by ligating oligonucleotides containing the wild-type or mutant putative target site of the CDK6 3′UTR into the multicloning site of the p-MIR Reporter Luciferase vector (Ambion). Cells were cotransfected using Lipofectamine 2000 (Invitrogen) with 0.4 μg of firefly luciferase reporter vector containing the wild type or mutant oligonucleotides, 0.02 μg pGal control vector, and 100 ng of miR-124a precursor. Luciferase activity was measured 48 hours after transfection using β-gal for normalization.

### REFERENCES

1. Miska, E et al. Microarray analysis of microRNA expression in the developing mammalian brain. Genome Biol. 5, R68 (2004).
2. Takai D, Jones P A. The CpG island searcher: a new WWW resource. In Silico Biol. 3, 235-240 (2003).
3. Ballestar, E et al. Methyl-CpG binding proteins identify novel sites of epigenetic inactivation in human cancer. EMBO J. 22, 6335-6345 (2003).

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all embodiments described herein are considered to be broadly applicable and combinable with any and all other consistent embodiments, as appropriate.

Various publications are cited herein, the disclosures of which are incorporated by reference in their entireties.

1. A method of diagnosing a disease in which an miRNA is expressed at a lower level comprising, in a test sample obtained from a subject, determining the methylation status of at least one gene encoding the miRNA in the sample, wherein the presence of methylation is indicative of the presence of the disease.

2. A method for predicting the likelihood of successful treatment or resistance to treatment of a disease in which an miRNA is expressed at a lower level with a DNA demethylating agent and/or a DNA methyltransferase inhibitor and/or a HDAC inhibitor comprising determining the methylation status of at least one gene encoding the miRNA in a sample obtained from a subject, wherein if the gene is methylated the likelihood of successful treatment is higher than if the gene is unmethylated and wherein if the gene is unmethylated the likelihood of resistance to treatment is higher than if the gene is methylated.

3. (canceled)

4. A method of selecting a suitable treatment regimen for a disease in which an miRNA is expressed at a lower level comprising determining the methylation status of at least one gene encoding the miRNA in a sample obtained from a subject, wherein if the gene is methylated a DNA demethylating agent and/or a DNA methyltransferase inhibitor and/or a HDAC inhibitor is selected for treatment.

5. The method of claim 1 wherein the methylation status of the gene in the test sample is compared to that of a control sample.

6. (canceled)

7. (canceled)

8. The method according to claim 1 wherein the disease is cancer.

9. The method according to claim 8 wherein the cancer is selected from colon cancer, breast cancer, lung cancer, leukaemia and lymphoma, preferably colon cancer.

10. The method according to claim 1 which comprises determining the methylation status of the gene or genes encoding 124a miRNA.

11. (canceled)

12. The method according to claim 1, wherein the methylation status is determined using a technique selected from methylation specific PCR, bisulphite sequencing, microarray techniques, real-time or end point amplification techniques and COBRA, either alone or in combination.

13. (canceled)

14. The method according to claim 12 wherein the methylation specific PCR comprises use of primer pairs selected from the primers comprising the nucleotide sequences set forth as SEQ ID NO: 2 and 3/6 and 7/10 and 11/or functional derivatives thereof in order to determine if the gene or genes encoding 124a miRNA is/are methylated.

15. The method according to claim 12 wherein the methylation specific PCR comprises use of primer pairs selected from the primers comprising the nucleotide sequences set forth as SEQ ID NO: 4 and 5/8 and 9/12 and 13 or functional derivatives thereof in order to determine if the gene or genes encoding 124a miRNA are unmethylated.

16. The method of claim 12 wherein bisulphite sequencing of the gene or genes encoding 124a miRNA is carried out by using sequencing primer pairs selected from the primers comprising the nucleotide sequences set forth as SEQ ID NO: 14 and 15/16 and 17/19 and 19 or functional derivatives thereof.

17. A method of diagnosing cancer comprising, in a test sample obtained from a subject, determining the expression levels or activity of 124a miRNA in the sample, wherein a reduced level of expression or activity is indicative of the presence of the disease.

18. A method for predicting the likelihood of successful treatment or resistance to treatment of cancer with a DNA demethylating agent and/or a DNA methyltransferase inhibitor and/or a HDAC inhibitor comprising determining the expression levels or activity of 124a miRNA in a test sample obtained from a subject, wherein a reduced level of expression or activity is indicative of the likelihood of successful treatment being higher than if the expression levels or activity are higher and wherein an increased level of expression or activity is indicative of the likelihood of successful treatment being lower than if the expression levels or activity are lower.

19. (canceled)

20. A method of selecting a suitable treatment regimen for cancer comprising determining the expression levels or activity of 124a miRNA in a test sample obtained from a subject, wherein if the expression levels or activity are lower a DNA demethylating agent and/or a DNA methyltransferase inhibitor and/or a HDAC inhibitor is selected for treatment.

21. The method of claim 17 wherein the expression levels of the 124a miRNA in the test sample is compared to that of a control sample.

22. (canceled)

23. (canceled)

24. The method according to claim 17 wherein the cancer is selected from colon cancer, breast cancer, lung cancer, leukaemia and lymphoma, preferably colon cancer.

25. (canceled)

26. (canceled)

27. (canceled)

28. (canceled)

29. (canceled)

30. (canceled)

31. (canceled)

32. (canceled)

33. A recombinant construct capable of expressing an miRNA comprising the nucleotide sequence set forth as SEQ ID NO: 1 or a functional derivative thereof with the ability to down-regulate CDK6 protein expression.

34. (canceled)

35. A method of treating a condition characterised by methylation of miRNA comprising administering to a patient in need of treatment a demethylating agent in an amount effective to restore miRNA function.

36. A method of treating a condition characterised by methylation of miRNA comprising administering to a patient in need of treatment one or more miRNAs, wherein the administered miRNAs complement miRNA function suppressed by methylation in the patient.

37. The method of claim 35 or 36 wherein the condition comprises cancer

38. The method of claim 37 wherein the cancer is selected from colon cancer, breast cancer, lung cancer, leukaemia and lymphoma, preferably colon cancer.

39. The method of claim 35 wherein the miRNA comprises 124a miRNA or a functional derivative thereof with the ability to down-regulate CDK6 (protein) expression.

40. A method of treating cancer in a patient comprising reducing CDK6 levels and/or activity.

41. The method of claim 40 wherein the cancer is selected from colon cancer, breast cancer, lung cancer, leukaemia and lymphoma, preferably colon cancer.

42. The method of claim 40 wherein the levels and/or activity of CDK6 are reduced by removing methylation of one or more genes encoding an miRNA molecule, which miRNA molecule acts to inhibit expression of CDK6 and/or by administering to the patient one or more miRNAs, wherein the administered miRNAs act to reduce the levels and/or activity of CDK6 in the patient.

43. The method of claim 42 wherein the one or more genes encoding an miRNA molecule whose methylation is removed comprises the one or more genes encoding 124a miRNA.

44. The method of claim 42 wherein the one or more miRNA molecules which is administered comprises 124a miRNA or a functional derivative thereof with the ability to down-regulate CDK6 protein expression.

45. A pharmaceutical composition comprising an miRNA together with a suitable carrier/excipient/diluent.

46. The composition of claim 45 wherein the miRNA comprises 124a miRNA or a functional derivative thereof with the ability to down-regulate CDK6 protein expression.

47. The composition of claim 45 which comprises a recombinant construct or a host cell or vector.

48. (canceled)

49. (canceled)

50. (canceled)

51. (canceled)

52. A kit for:
(a) diagnosing a disease in which an miRNA is expressed at a lower level; and/or
(b) predicting the likelihood of successful treatment of a disease in which an miRNA is expressed at a lower level and/or the likelihood of resistance to treatment of a disease in which an miRNA is expressed at a lower level with a DNA demethylating agent and/or a DNA methyltransferase inhibitor and/or a HDAC inhibitor, and/or
(c) selecting a suitable treatment regimen for a disease in which an miRNA is expressed at a lower level
comprising carrier means containing therein a set of primers for use in detecting the methylation status of at least one gene encoding the miRNA or for use in detecting the expression level of an miRNA which is expressed at a lower level in the disease.

53. The kit according to claim 52 wherein the miRNA is 124a miRNA.

54. The kit according to claim 53 for use in bisulphite sequencing of one or more genes encoding 124a miRNA comprising at least one sense and antisense primer pair selected from the primers comprising the nucleotide sequences set forth as SEQ ID NO 14, 15, 16, 17, 18 and 19, and functional derivatives thereof which are useful in bisulfite sequencing of one or more genes encoding 124a miRNA.

55. The kit according to claim 53 comprising at least one sense and antisense primer pair selected from the primers comprising the nucleotide sequences set forth as SEQ ID NO: 2, 3, 4, 5, 6, 7, 8, 9, 10, 11, 12 and 13 and functional derivatives thereof which are useful in MSP for determining the methylation status of one or more genes encoding 124a miRNA.

56. (canceled)

57. The kit according to claim 52 comprising a bisulphite reagent.

58. (canceled)

59. (canceled)

60. The kit according to claim 52 for use in RT-PCR comprising at least one sense and antisense primer pair selected from the primers comprising the nucleotide sequences set forth as SEQ ID NO: 32 and 33 or SEQ ID NO: 37 and 38 respectively and functional derivatives thereof which are useful in RT-PCR for determining expression levels of 124a miRNA.

61. (canceled)


SEQUENCE LISTING (47 sequences)

SEQ ID NO: 1 - RNA, 22 nt, Homo sapiens
accguaaguggcgcacggaauu

SEQ ID NO: 2 - DNA, 21 bp, Artificial sequence
  Sense MSP primer
aaagagtttttggaagacgtc

SEQ ID NO: 3 - DNA, 20 bp, Artificial sequence
  Antisense MSP primer
aataaaaaacgacgcgtata

SEQ ID NO: 4 - DNA, 24 bp, Artificial sequence
  Sense MSP primer
aataaagagtttttggaagatgtt

SEQ ID NO: 5 - DNA, 26 bp, Artificial sequence
  Antisense MSP primer
aaaaaaataaaaaacaacacatatac

SEQ ID NO: 6 - DNA, 24 bp, Artificial sequence
  Sense MSP primer
gggtaattaatttggatttacgtc

SEQ ID NO: 7 - DNA, 24 bp, Artificial sequence
  Antisense MSP primer
accgctattaattaatctattccg

SEQ ID NO: 8 - DNA, 25 bp, Artificial sequence
  Sense MSP primer
ggggtaattaatttggatttatgtt

SEQ ID NO: 9 - DNA, 27 bp, Artificial sequence
  Antisense MSP primer
aaaaccactattaattaatctattcca

SEQ ID NO: 10 - DNA, 21 bp, Artificial sequence
  Sense MSP primer
gcgaggattttacgtaagttc

SEQ ID NO: 11 - DNA, 21 bp, Artificial sequence
  Antisense MSP primer
ccgcgtaccttaattatataa

SEQ ID NO: 12 - DNA, 23 bp, Artificial sequence
  Sense MSP primer
gggtgaggattttatgtaagttt

SEQ ID NO: 13 - DNA, 27 bp, Artificial sequence
  Antisense MSP primer
ttcaccacataccttaattatataaac

SEQ ID NO: 14 - DNA, 25 bp, Artificial sequence
  Bisulphite sequencing primer
aaggatgggggagaataaagagttt

SEQ ID NO: 15 - DNA, 25 bp, Artificial sequence
  Bisulphite sequencing primer
ctcaaccaaccccattcttaacatt

SEQ ID NO: 16 - DNA, 26 bp, Artificial sequence
  Bisulphite sequencing primer
ggtaatggttatgayggagaatatgt

SEQ ID NO: 17 - DNA, 24 bp, Artificial sequence
  Bisulphite sequencing primer
ccaactcctatctctactcatctc

SEQ ID NO: 18 - DNA, 24 bp, Artificial sequence
  Bisulphite sequencing primer
ggaaaggggagaagtgtgggtttt

SEQ ID NO: 19 - DNA, 25 bp, Artificial sequence
  Bsiulphite sequencing primer
raaaacrcctctcttaacattcacc

SEQ ID NO: 20 - DNA, 26 bp, Artificial sequence
  Bisulphite sequencing primer
ggaagttgagtttttagtgagttgag

SEQ ID NO: 21 - DNA, 21 bp, Artificial sequence
  Bisulphite sequencing primer
aattctcctactaccrcctcc

SEQ ID NO: 22 - DNA, 22 bp, Artificial sequence
  Bisulphite sequencing primer
ttggggaagggaagggggtttt

SEQ ID NO: 23 - DNA, 23 bp, Artificial sequence
  Bisulphite sequencing primer
cctacctcaacctcccaaataac

SEQ ID NO: 24 - DNA, 16 bp, Artificial sequence
  Primer
ccttaattgtatggac

SEQ ID NO: 25 - DNA, 16 bp, Artificial sequence
  Primer
attaaatcaaggtccg

SEQ ID NO: 26 - DNA, 16 bp, Artificial sequence
  Primer
attaaatcaaggtccg

SEQ ID NO: 27 - DNA, 20 bp, Artificial sequence
  Primer
ccgctgtgaacacggagaga

SEQ ID NO: 28 - DNA, 22 bp, Artificial sequence
  Primer
gaacacggagagcagagcctct

SEQ ID NO: 29 - DNA, 22 bp, Artificial sequence
  Primer
ggtccgctgtgaacacgcagag

SEQ ID NO: 30 - DNA, 20 bp, Artificial sequence
  Primer
gaggggtgggtagaagatgg

SEQ ID NO: 31 - DNA, 22 bp, Artificial sequence
  Primer
gtccgctgtgaacacggagagc

SEQ ID NO: 32 - DNA, 20 bp, Artificial sequence
  Primer
gttcacagcggaccttgatt

SEQ ID NO: 33 - DNA, 20 bp, Artificial sequence
  Primer
accgcgtgccttaattgtat

SEQ ID NO: 34 - DNA, 20 bp, Artificial sequence
  Primer
gaagatctcaggcagtgacc

SEQ ID NO: 35 - DNA, 25 bp, Artificial sequence
  Primer
aaacagtaacactctaaaaggatgc

SEQ ID NO: 36 - DNA, 46 bp, Artificial sequence
  poly(T) adaptor
gcgagcacagaattaatacgactcactataggttttttttttttvn

SEQ ID NO: 37 - DNA, 24 bp, Artificial sequence
  Primer
gcgagcacagaattaatacgactc

SEQ ID NO: 38 - DNA, 22 bp, Artificial sequence
  Primer
ttaaggcacgcggtgaatgcca

SEQ ID NO: 39 - DNA, 22 bp, Artificial sequence
  Primer
atcgtgcatccttttagagtgt

SEQ ID NO: 40 - DNA, 20 bp, Artificial sequence
  Primer
caaagagcctttggaagacg

SEQ ID NO: 41 - DNA, 20 bp, Artificial sequence
  Primer
ggaagaggggtgggtagaag

SEQ ID NO: 42 - DNA, 20 bp, Artificial sequence
  Primer
gcgtggtccttaaaaacctg

SEQ ID NO: 43 - DNA, 20 bp, Artificial sequence
  Primer
ccatgccatttacagcacac

SEQ ID NO: 44 - DNA, 19 bp, Artificial sequence
  Primer
ggagaagtgtgggctcctc

SEQ ID NO: 45 - DNA, 20 bp, Artificial sequence
  Primer
aatcaaggtccgctgtgaac

SEQ ID NO: 46 - DNA, 20 bp, Artificial sequence
  Primer
gcctatgggaaggtgttcaa

SEQ ID NO: 47 - DNA, 20 bp, Artificial sequence
  Primer
cactccaggctctggaactt